(19) United States
(12) Patent Application Publication
(10) Pub. No.: US 2005/0153295 A1
(43) Pub. Date: Jul. 14, 2005

(54) Diagnostic and therapeutic use of human maguin proteins and nucleic acids for neurodegenerative diseases

(76) Inventors: Heinz Von Der Kammer, Hamburg (DE); Jozef Hanes, Schenefeld (DE); Johannes Pohlner, Hamburg (DE)

Correspondence Address: JACOBSON HOLMAN PLLC, 400 SEVENTH STREET N.W., SUITE 600, WASHINGTON, DC, 20004, US

(21) Appl. No.: 10/508,343

(22) PCT Filed: Mar. 19, 2003
(86) PCT No.: PCT/EP03/02857

Related U.S. Application Data
(60) Provisional application No. 60/365,815, filed on Mar. 21, 2002.

(30) Foreign Application Priority Data
Mar. 21, 2002 (EP) 02006353.3

(51) Int. Cl. C12Q 1/68; G01N 33/53; G01N 33/567

(52) U.S. Cl. 435/6; 435/7.2

(57) ABSTRACT

The present invention discloses the differential expression of the human MAGUIN-1 and/or human MAGUIN-2 gene in specific brain regions of Alzheimer's disease patients. Based on this finding, this invention provides a method for diagnosing or prognosticating a neurodegenerative disease, in particular Alzheimer's disease, in a subject, or for determining whether a subject is at increased risk of developing such a disease. Furthermore, this invention provides therapeutic and prophylactic methods for treating or preventing Alzheimer's disease and related neurodegenerative disorders using a human MAGUIN gene. A method of screening for modulating agents of neurodegenerative diseases is also disclosed.

The present invention relates to methods of diagnosing, prognosticating and monitoring the progression of neurodegenerative diseases in a subject. Furthermore, methods of therapy control and screening for modulating agents of neurodegenerative diseases are provided. The invention also discloses pharmaceutical compositions, kits, and recombinant animal models.

Neurodegenerative diseases, in particular Alzheimer's disease (AD), have a strongly debilitating impact on a patient's life. Furthermore, these diseases constitute an enormous health, social, and economic burden. AD is the most common neurodegenerative disease, accounting for about 70% of all dementia cases, and it is probably the most devastating age-related neurodegenerative condition affecting about 10% of the population over 65 years of age and up to 45% over age 85 (for a recent review see Vickers et al., Progress in Neurobiology 2000, 60: 139-165). Presently, this amounts to an estimated 12 million cases in the US, Europe, and Japan. This situation will inevitably worsen with the demographic increase in the number of old people (“aging of the baby boomers”) in developed countries. The neuropathological hallmarks that occur in the brains of individuals with AD are senile plaques, composed of amyloid-β protein, and profound cytoskeletal changes coinciding with the appearance of abnormal filamentous structures and the formation of neurofibrillary tangles.

The amyloid-β (Aβ) protein evolves from the cleavage of the amyloid precursor protein (APP) by different kinds of proteases. The cleavage by the β/γ-secretase leads to the formation of Aβ peptides of different lengths, typically a short more soluble and slow aggregating peptide consisting of 40 amino acids and a longer 42 amino acid peptide, which rapidly aggregates outside the cells, forming the characteristic amyloid plaques (Selkoe, Physiological Rev 2001, 81: 741-66; Greenfield et al., Frontiers Bioscience 2000, 5: D72-83). Two types of plaques, diffuse plaques and neuritic plaques, can be detected in the brain of AD patients, the latter ones being the classical, most prevalent type. They are primarily found in the cerebral cortex and hippocampus. The neuritic plaques have a diameter of 50 μm to 200 μm and are composed of insoluble fibrillar amyloids, fragments of dead neurons, of microglia and astrocytes, and other components such as neurotransmitters, apolipoprotein E, glycosaminoglycans, α1-antichymotrypsin and others. The generation of toxic Aβ deposits in the brain starts very early in the course of AD, and it is discussed to be a key player for the subsequent destructive processes leading to AD pathology. The other pathological hallmarks of AD are neurofibrillary tangles (NFTs) and abnormal neurites, described as neuropil threads (Braak and Braak, Acta Neuropathol 1991, 82: 239-259). NFTs emerge inside neurons and consist of chemically altered tau, which forms paired helical filaments twisted around each other. Along the formation of NFTs, a loss of neurons can be observed. It is discussed that said neuron loss may be due to a damaged microtubule-associated transport system (Johnson and Jenkins, J Alzheimers Dis 1996, 1: 38-58; Johnson and (1) Hartigan, J Alzheimers Dis 1999, 1: 329-351). The appearance of neurofibrillary tangles and their increasing number correlates well with the clinical severity of AD (Schmitt et al., Neurology 2000, 55: 370-376).

AD is a progressive disease that is associated with early deficits in memory formation and ultimately leads to the complete erosion of higher cognitive function. The cognitive disturbances include among other things memory impairment, aphasia, agnosia and the loss of executive functioning. A characteristic feature of the pathogenesis of AD is the selective vulnerability of particular brain regions and subpopulations of nerve cells to the degenerative process. Specifically, the temporal lobe region and the hippocampus are affected early and more severely during the progression of the disease. On the other hand, neurons within the frontal cortex, occipital cortex, and the cerebellum remain largely intact and are protected from neurodegeneration (Terry et al., Annals of Neurology 1981, 10: 184-92).

The age of onset of AD may vary within a range of 50 years, with early-onset AD occurring in people younger than 65 years of age, and late-onset of AD occurring in those older than 65 years. About 10% of all AD cases suffer from early-onset AD, with only 1-2% being familial, inherited cases.

Currently, there is no cure for AD, nor is there an effective treatment to halt the progression of AD or even to diagnose AD ante-mortem with high probability. Several risk factors have been identified that predispose an individual to develop AD, among them most prominently the epsilon 4 allele of the three different existing alleles (epsilon 2, 3, and 4) of the apolipoprotein E gene (ApoE) (Strittmatter et al., Proc Natl Acad Sci USA 1993, 90: 1977-81; Roses, Ann NY Acad Sci 1998, 855: 738-43). The polymorphic plasmaprotein ApoE plays a role in the intercellular cholesterol and phospholipid transport by binding low-density lipoprotein receptors, and it seems to play a role in neurite growth and regeneration. Efforts to detect further susceptibility genes and disease-linked polymorphisms, lead to the assumption that specific regions and genes on human chromosomes 10 and 12 may be associated with late-onset AD (Myers et al., Science 2000, 290: 2304-5; Bertram et al., Science 2000, 290: 2303; Scott et al., Am J Hum Genet 2000, 66: 922-32).

Although there are rare examples of early-onset AD which have been attributed to genetic defects in the genes for amyloid precursor protein (APP) on chromosome 21, presenilin-1 on chromosome 14, and presenilin-2 on chromosome 1, the prevalent form of late-onset sporadic AD is of hitherto unknown etiologic origin. The mutations found to date account for only half of the familial AD cases, which is less than 2% of all AD patients. The late onset and complex pathogenesis of neurodegenerative disorders pose a formidable challenge to the development of therapeutic and diagnostic agents. It is pivotal to expand the pool of potential drug targets and diagnostic markers. It is therefore an object of the present invention to provide insight into the pathogenesis of neurological diseases and to provide methods, materials, agents, compositions, and animal models which are suited inter alia for the diagnosis and development of a treatment of these diseases. This object has been solved by the features of the independent claims. The subclaims define preferred embodiments of the present invention.

Neurons conduct signals in the form of electrical impulses. The communication between the cells of a neuronal network involves several chemical steps, including the production, release, and transport of signal molecules and the recognition of such messengers by a receptor. These steps take place at the anatomical contact of an axon with the dendrites (axonodendritic), the cell body (axonosomatic), or rarely with the axon of another neuron, and also between neurons and cells of muscle- and gland tissue. Trillions of such specialized cell junctions (synapses) in the human brain are crucial for controlling mental activity and learning processes (Tessier-Lavigne and Goodman, Science 1996, 274: 1123-1133). A synapse is composed of the presynaptic element, the synaptic cleft with a spacing distance of about 20-30 nm and the postsynaptic component. They are responsible for altering the membrane potential of the postsynaptic neuron or other effector cells. The quality and intensity of information transferred relies on the number, location, and distribution of synapses. The basis of learning and memory is believed to be due to brain plasticity, i.e. to the plasticity of synapses. The storage and processing of information cause alterations in the structure, chemistry, and strength of synapses and the formation of new synapses (Poirazi and Mel, Neuron 2001, 29: 779-796). The postsynaptic density (PSD) is a specialized synaptic signaling assemblage, composed of a specific set of proteins which are assembled together and linked to the cytoplasmic face of the postsynaptic membrane. An important function of the postsynaptic density is the provision of a structural matrix consisting of cytoskeletal and regulatory proteins which localize and accumulate neurotransmitter receptors (e.g. glutamate receptors) and anchor signaling molecules at the postsynaptic membrane (Sheng and Kim, Current Opinion Neurobiology 1996, 6: 602-608). Neurotransmitter receptors convert the extracellular chemical signals into intracellular signals. Neurotransmitters are released from the presynaptic membrane into the synaptic cleft via exocytotic processes (vesicle formation). At the postsynaptic membrane they are bound by their specific receptors, resulting in the generation of second messengers. Thus, neurotransmitters function as mediators of nerve impulse transmission across the synapse. The PSD organizes and regulates postsynaptic signal transduction (Kim and Huganir, Current Opinion Cell Biology 1999, 11:248-254). To date, several components of the PSD have been identified, among them the prototypic synaptic scaffolding protein postsynaptic density (PSD)-95/synapse-associated protein (SAP) 90. PSD-95/SAP90 and its isoforms belong to a family of membrane-associated guanylate kinases (MAGUK) (Hirao et al., Journal of Biological Chemistry 1998, 273: 21105-21110; Hirao et al., Journal of Biological Chemistry 2000, 275: 2966-2972). The members of the MAGUK protein family are multiple PDZ-domain containing proteins which interact with many neuronal adhesion proteins, receptors (e.g. N-methyl-D-aspartate (NMDA)) and ion-channels (e.g. potassium channels) through these domains and mediate their assembling at the PSD (Kim et al., Neuron 1996, 17: 103-113).

In this context, a novel neuronal membrane-associated guanylate kinase-interacting protein, denoted MAGUIN, was found by Yao et al. (Journal of Biological Chemistry 1999, 274: 11889-11896). Using the PDZ-domain sequence of the neurospecific synaptic scaffolding molecule (S-SCAM) as bait, the authors screened a rat brain yeast two-hybrid library and obtained several positive clones, among them two clones subsequently named rat MAGUIN-1 and rat MAGUIN-2 (GenBank accession numbers AF102853 and AF102854, respectively). A Northern blot analysis of different rat tissues with a rat MAGUIN-1 probe revealed brain specific hybridization signals at 4.4 kDa and 5.4 kDa. MAGUIN proteins have a chimerical molecular structure consisting of several protein modules, an N-terminally located sterile alpha-motif (SAM) (aa 8-75), a PDZ-domain (aa 156-296) and a C-terminally located Pleckstrin-homology (PH)-domain (aa 571-667). The Sterile Alpha Motif (SAM) contains four different domain structures, spanning approximately 70 amino acids generating a compact five-helix bundle with a highly conserved hydrophobic core. This motif was found to be part of a number of types of proteins, including signal transduction proteins, playing a role in mediating protein-protein interactions or DNA binding (Schultz et al., Protein Science 1997, 6: 249-253). The term PDZ is named after three proteins (i.e. PSD-95/SAP90, Drosophila discs-large tumor suppressor protein (Dlg-A) and the tight junction protein ZO-1), originally identified as proteins sharing the same repeats of about 90 amino acids. These amino acids form a distinctive structure of two α helices and six β sheets which mediate the interaction with the carboxyl termini of various proteins. Often, PDZ-domains of different proteins can heterodimerize with each other. The target proteins are transmembrane receptors, ion channels, or signaling proteins. PDZ-domain binding seems to be important in receptor clustering and in recruiting signal transduction molecules to the plasma membrane. PDZ-domain harboring proteins are for example tyrosine phosphatases and the previously described membrane-associated guanylate kinase-like proteins (Doyle et al., Cell 1996, 85: 1067-1076). The Pleckstrin-homology (PH)-domain forms an anti-parallel perpendicular β-sheet sandwich with a succeeding α-helical structure. The β-sheet-loops are important for high affinity binding to specific phosphatidylinositide phosphates, allowing signaling proteins containing the PH-domain to anchor to membranes, for example as studied for GTP binding proteins, protein kinases and phospholipase C isoforms (Lemmon et al., Trends Cell Biology 1997, 7: 237-242).

Rat MAGUIN-1 and rat MAGUIN-2 cover 3099 and 2691 nucleotides of coding sequence, respectively, (GenBank accession numbers AF102853, AF102854) which encode for proteins of 1032 aa amino acids (rat MAGUIN-1) (GenBank accession number aad04568) and 896 amino acids (rat MAGUIN-2) (GenBank accession number aad04567), respectively. Rat MAGUIN-2 lacks the 3′ terminal PDZ binding motif of rat MAGUIN-1. In neurons, the rat MAGUIN proteins are localized in the cell body and in neurites where they are associated with the plasma-membrane via their PH-domains. Full-length rat MAGUIN-1 could be recovered from neuronal membrane fractions. Rat MAGUIN-1, but not rat MAGUIN-2, interacts via the PDZ-binding motif with the PDZ-domain of PSD-95/SAP90 and the synaptic scaffolding molecule (S-SCAM) (Hirao et al., Journal of Biological Chemistry 1998, 273: 21105-21110). S-SCAM has recently been identified as a multiple PDZ-domain containing protein, interacting with SAP90/PSD-95-associated protein (SAPAP), the NMDA receptor, and neuronal adhesion molecules. Additionally, interaction of the PH-domain of rat MAGUIN-1 with the kinase domain of Raf could be confirmed in vitro and in vivo, but their is no evidence for the activation of Raf or its recruitment to the plasma membrane by rat MAGUIN-1.

To date, the precise function of rat MAGUIN-1 and rat MAGUIN-2 is still not clear. Some clues for the role of rat MAGUIN-1 in the cellular context come from particular protein domains and motifs, specific brain expression patterns, and structural homologies to other proteins like CNK (connector enhancer of kinase suppressor of ras (ksr)), as described for the fruit fly. CNK binds kinase suppressor for Ras and Raf kinase, functions in the Ras/MAP kinase pathway, and has been found to play a profound role in the regulation of eye development (Therrien et al., Cell 1998, 95: 343-353). On the basis of the similarities between Drosophila CNK (contains SAM, PDZ and PH-domains) and rat MAGUIN-1, rat MAGUIN-1 is discussed as a rodent homolog to CNK. Thus, it is likely that rat MAGUIN-1 assembles components of synaptic junctions and regulators of the MAP kinase pathway and links them to NMDA receptors and neuronal adhesion molecules. To date, no experiments have been described that show a relationship between a differential expression of human MAGUIN genes, neither on a transcriptional nor on a translational level, and the pathology of neurodegenerative disorders.

The disclosure in the present invention of the human MAGUIN-1 gene and the identification of a link of both human MAGUIN-1 and/or human MAGUIN-2 to neurodegenerative diseases, particularly Alzheimer's disease, offers new ways, inter alia, for the diagnosis and treatment of such diseases.

The singular forms “a”, “an”, and “the” as used herein and in the claims include plural reference unless the context dictates otherwise. For example “a cell” means as well a plurality of cells, and so forth. The term “and/or” as used in the present specification and in the claims implies that the phrases before and after this term are to be considered either as alternatives or in combination. For instance, the wording “determination of a level and/or an activity” means that either only a level, or only an activity, or both a level and an activity are determined. The term “level” as used herein is meant to comprise a gage of, or a measure of the amount of, or a concentration of a transcription product, for instance an mRNA, or a translation product, for instance a protein or polypeptide. The term “activity” as used herein shall be understood as a measure for the ability of a transcription product or a translation product to produce a biological effect or a measure for a level of biologically active molecules. The term “activity” also refers to enzymatic activity. The terms “level” and/or “activity” further refer to gene expression levels or gene activity. Gene expression can be defined as the utilization of the information contained in a gene by transcription and translation leading to the production of a gene product. “Dysregulation” shall mean an upregulation or downregulation of gene expression. A gene product comprises either RNA or protein and is the result of expression of a gene. The amount of a gene product can be used to measure how active a gene is. The term “gene” as used in the present specification and in the claims comprises both coding regions (exons) as well as non-coding regions (e.g. non-coding regulatory elements such as promotors or enhancers, introns, leader and trailer sequences). The term “ORF” is an acronym for “open reading frame” and refers to a nucleic acid sequence that does not possess a stop codon in at least one reading frame and therefore can potentially be translated into a sequence of amino acids. “Regulatory elements” as used in the present disclosure shall comprise inducible and non-inducible promotors, enhancers, operators and other elements that drive and regulate gene expression. The term “fragment” as used herein is meant to comprise e.g. an alternatively spliced, or truncated, or otherwise cleaved transcription product or translation product. The term “derivative” as used herein refers to a mutant, or an RNA-edited, or a chemically modified, or otherwise altered transcription product, or to a mutant, or chemically modified, or otherwise altered translation product. For instance, a “derivative” may be generated by processes such as altered phosphorylation, or glycosylation, or acetylation, or lipidation, or by altered signal peptide cleavage or other types of maturation cleavage. These processes may occur post-translationally. The term “modulator” as used in the present invention and in the claims refers to a molecule capable of changing or altering the level and/or the activity of a gene, or a transcription product of a gene, or a translation product of a gene. Preferably, a “modulator” is capable of changing or altering the biological activity of a transcription product or a translation product of a gene. Said modulation, for instance, may be an increase or a decrease in enzyme activity, a change in binding characteristics, or any other change or alteration in the biological, functional, or immunological properties of said translation product of a gene.

The terms “agent”, “reagent”, or “compound” refer to any substance, chemical, composition or extract that have a positive or negative biological effect on a cell, tissue, body fluid, or within the context of any biological system, or any assay system examined. They can be agonists, antagonists, partial agonists or inverse agonists of a target. Such agents, reagents, or compounds may be nucleic acids, natural or synthetic peptides or protein complexes, or fusion proteins. They may also be antibodies, organic or anorganic molecules or compositions, small molecules, drugs and any combinations of any of said agents above. They may be used for testing, for diagnostic or for therapeutic purposes. The terms “oligonucleotide primer” or “primer” refer to short nucleic acid sequences which can anneal to a given target polynucleotide by hybridization of the complementary base pairs and can be extended by a polymerase. They may be chosen to be specific to a particular sequence or they may be randomly selected, e.g. they will prime all possible sequences in a mix. The length of primers used herein may vary from 10 nucleotides to 80 nucleotides. “Probes” are short nucleic acid sequences of the nucleic acid sequences described and disclosed herein or sequences complementary therewith. They may comprise full length sequences, or fragments, derivatives, isoforms, or variants of a given sequence. The identification of hybridization complexes between a “probe” and an assayed sample allows the detection of the presence of other similar sequences within that sample. As used herein, “homolog or homology” is a term used in the art to describe the relatedness of a nucleotide or peptide sequence to another nucleotide or peptide sequence, which is determined by the degree of identity and/or similarity between said sequences compared.

The term “variant” as used herein refers to any polypeptide and protein, in reference to polypeptides and proteins disclosed in the present invention, in which one or more amino acids are added and/or substituted and/or deleted and/or inserted at the N-terminus, and/or the C-terminus, and/or within the native amino acid sequences of the native polypeptides or proteins of the present invention. Furthermore, the term “variant” shall include any shorter or longer version of a polypeptide or protein. “Variants” shall also comprise a sequence that has at least about 80% sequence identity, more preferably at least about 90% sequence identity, and most preferably at least about 95% sequence identity with the amino acid sequences of SEQ ID NO. 1 and/or SEQ ID NO. 2, respectively. Derivatives, variants, and fragments may include, but are not limited to, a functional SAM, a functional PDZ and a functional PH domain or other functional modules within the polypeptide sequence of human MAGUIN-1 and/or human Maguin-2 proteins. “Variants” of a protein molecule shown in SEQ ID NO. 1 and/or SEQ ID NO. 2 may include, for example, proteins with conservative amino acid substitutions in highly conservative regions. For example, isoleucine, valine and leucine can each be substituted for one another. Aspartate and glutamate can be substituted for each other. Glutamine and asparagine can be substituted for each other. Serine and threonine can be substituted for each other. Amino acid substitutions in less conservative regions include, for example, isoleucine, valine and leucine, which can each be substituted for one another. Aspartate and glutamate can be substituted for each other. Glutamine and asparagine can be subsituted for each other. Serine and threonine can be substituted for each other. Glycine and alanine can be substituted for each other. Alanine and valine can be substituted for each other. Methionine can be substituted for each of leucine, isoleucine or valine, and vice versa. Lysine and arginine can be substituted for each other. One of aspartate and glutamate can be substituted for one of arginine or lysine, and vice versa. Histidine can be substituted for arginine or lysine, and vice versa. Glutamine and glutamate can be substituted for each other. Asparagine and aspartate can be substituted for each other. Other examples of protein modifications include glycosylation and further post-translational modifications. “Proteins and polypeptides” of the present invention include variants, fragments and chemical derivatives of the protein comprising SEQ ID NO. 1 and/or SEQ ID NO. 2. They can include proteins and polypeptides which can be isolated from nature or which can be produced by recombinant and/or synthetic means. Native proteins or polypeptides refer to naturally-occurring truncated or secreted forms, naturally occurring variant forms (e.g. splice variants) and naturally occurring allelic variants. As used herein, protein and polypeptide refers to a linear series of amino acid residues connected to one another by peptide bonds between the alpha-amino group and carboxy groups of adjacent amino acid residues. Other covalent bonds, such as amide and disulfide bonds, may also be present. The term “isolated” as used herein is considered to refer to molecules that are removed from their natural environment, i.e. isolated from a cell or from a living organism in which they normally occur, and that are separated or essentially purified from the coexisting components with which they are found to be associated in nature. This notion further means that the sequences encoding such molecules can be linked by the hand of man to polynucleotides, to which they are not linked in their natural state, and that such molecules can be produced by recombinant and/or synthetic means. Even if for said purposes those sequences may be introduced into living or non-living organisms by methods known to those skilled in the art, and even if those sequences are still present in said organisms, they are still considered to be isolated. In the present invention, the terms “risk”, “susceptibility” and “predisposition” are tantamount and are used with respect to the probability of developing a neurodegenerative disease, preferably Alzheimer's disease.

The term ‘AD’ shall mean Alzheimer's disease. “AD-type neuropathology” as used herein refers to neuropathological, neurophysiological, histopathological and clinical hallmarks as described in the instant invention and as commonly known from state-of-the-art literature (see: Iqbal, Swaab, Winblad and Wisniewski, Alzheimer's Disease and Related Disorders (Etiology, Pathogenesis and Therapeutics), Wiley & Sons, New York, Weinheim, Toronto, 1999; Scinto and Daffner, Early Diagnosis of Alzheimer's Disease, Humana Press, Totowa, N.J., 2000; Mayeux and Christen, Epidemiology of Alzheimer's Disease: From Gene to Prevention, Springer Press, Berlin, Heidelberg, N.Y., 1999; Younkin, Tanzi and Christen, Presenilins and Alzheimer's Disease, Springer Press, Berlin, Heidelberg, N.Y., 1998).

Neurodegenerative diseases or disorders according to the present invention comprise Alzheimer's disease, Parkinson's disease, Huntington's disease, amyotrophic lateral sclerosis, Pick's disease, fronto-temporal dementia, progressive nuclear palsy, corticobasal degeneration, cerebro-vascular dementia, multiple system atrophy, argyrophilic grain dementia and other tauopathies, and mild-cognitive impairment. Further conditions involving neurodegenerative processes are, for instance, age-related macular degeneration, narcolepsy, motor neuron diseases, prion diseases, traumatic nerve injury and repair, and multiple sclerosis.

In one aspect, the invention features a method of diagnosing or prognosticating a neurodegenerative disease in a subject, or determining whether a subject is at increased risk of developing said disease. The method comprises: determining a level, or an activity, or both said level and said activity of (i) a transcription product of a gene coding for human MAGUIN-1 and/or human MAGUIN-2, and/or of (ii) a translation product of a gene coding for human MAGUIN-1 and/or human MAGUIN-2, and/or of (iii) a fragment, or derivative, or variant of said transcription or translation product in a sample from said subject and comparing said level, and/or said activity to a reference value representing a known disease or health status, thereby diagnosing or prognosticating said neurodegenerative disease in said subject, or determining whether said subject is at increased risk of developing said neurodegenerative disease.

The invention also relates to the construction and the use of primers and probes which are unique to the nucleic acid sequences, or fragments, or variants thereof, as disclosed in the present invention. The oligonucleotide primers and/or probes can be labeled specifically with fluorescent, bioluminescent, magnetic, or radioactive substances. The invention further relates to the detection and the production of said nucleic acid sequences, or fragments and variants thereof, using said specific oligonucleotide primers in appropriate combinations. PCR-analysis, a method well known to those skilled in the art, can be performed with said primer combinations to amplify said gene specific nucleic acid sequences from a sample containing nucleic acids. Such sample may be derived either from healthy or diseased subjects. Whether an amplification results in a specific nucleic acid product or not, and whether a fragment of different length can be obtained or not, may be indicative for a neurodegenerative disease, in particular Alzheimer's disease. Thus, the invention provides nucleic acid sequences, oligonucleotide primers, and probes of at least 10 bases in length up to the entire coding and gene sequences, useful for the detection of gene mutations and single nucleotide polymorphisms in a given sample comprising nucleic acid sequences to be examined, which may be associated with neurodegenerative diseases, in particular Alzheimer's disease. This feature has utility for developing rapid DNA-based diagnostic tests, preferably also in the format of a kit.

In a further aspect, the invention features a method of monitoring the progression of a neurodegenerative disease in a subject. A level, or an activity, or both said level and said activity, of (i) a transcription product of a gene coding for human MAGUIN-1 and/or human MAGUIN-2, and/or of (ii) a translation product of a gene coding for human MAGUIN-1 and/or human MAGUIN-2, and/or of (iii) a fragment, or derivative, or variant of said transcription or translation product in a sample from said subject is determined. Said level and/or said activity is compared to a reference value representing a known disease or health status. Thereby the progression of said neurodegenerative disease in said subject is monitored.

In still a further aspect, the invention features a method of evaluating a treatment for a neurodegenerative disease, comprising, determining a level, or an activity, or both said level and said activity of (i) a transcription product of a gene coding for human MAGUIN-1 and/or human MAGUIN-2, and/or of (ii) a translation product of a gene coding for human MAGUIN-1 and/or human MAGUIN-2, and/or of (iii) a fragment; or derivative, or variant of said transcription or translation product in a sample obtained from a subject being treated for said disease. Said level, or said activity, or both said level and said activity are compared to a reference value representing a known disease or health status, thereby evaluating the treatment for said neurodegenerative disease.

In a preferred embodiment of the herein claimed methods, kits, recombinant animals, molecules, assays, and uses of the instant invention, said gene coding for the membrane-associated guanylate kinase-interacting proteins is the gene coding for the human neuronal membrane-associated guanylate kinase-interacting protein 1 (SEQ ID NO. 5), also termed (MAGUIN-1), and the human neuronal membrane-associated guanylate kinase-interacting protein 2 (SEQ ID NO. 6).

In a further preferred embodiment of the herein claimed methods, kits, recombinant animals, molecules, assays, and uses of the instant invention, said neurodegenerative disease or disorder is Alzheimer's disease, and said subjects suffer from Alzheimer's disease.

The present invention discloses the differential expression and regulation of the human MAGUIN-1 and/or human MAGUIN-2 gene in specific brain regions of Alzheimer's disease patients. Consequently, human MAGUIN-1 and/or human MAGUIN-2 and their corresponding translation products may have a causative role in the regional selective neuronal degeneration typically observed in Alzheimer's disease. Alternatively, human MAGUIN-1 and/or human MAGUIN-2 may confer a neuroprotective function to the remaining surviving nerve cells. Based on these disclosures, the present invention has utility for the diagnostic evaluation and prognosis as well as for the identification of a predisposition to a neurodegenerative disease, in particular Alzheimer's disease. Furthermore, the present invention provides methods for the diagnostic monitoring of patients undergoing treatment for such a disease.

It is preferred that the sample to be analyzed and determined is selected from the group comprising brain tissue, or other tissues, or other body cells. The sample can also comprise cerebrospinal fluid or other body fluids including saliva, urine, serum plasma, or mucus. Preferably, the methods of diagnosis, prognosis, monitoring the progression or evaluating a treatment for a neurodegenerative disease, according to the instant invention, can be practiced ex corpore, and such methods preferably relate to samples, for instance, body fluids or cells, removed, collected, or isolated from a subject or patient.

In further preferred embodiments, said reference value is that of a level, or an activity, or both said level and said activity of (i) a transcription product of a gene coding for human MAGUIN-1 and/or human MAGUIN-2, and/or of (ii) a translation product of a gene coding for human MAGUIN-1 and/or human MAGUIN-2, and/or of (iii) a fragment, or derivative, or variant of said transcription or translation product in a sample from a subject not suffering from said neurodegenerative disease.

In preferred embodiments, an alteration in the level and/or activity of a transcription product of the gene coding for human MAGUIN-1 and/or human MAGUIN-2 and/or a translation product of the gene coding for human MAGUIN-1 and/or human MAGUIN-2 protein in a sample cell, or tissue, or body fluid from said subject relative to a reference value representing a known health status indicates a diagnosis, or prognosis, or increased risk of becoming diseased with a neurodegenerative disease, particularly Alzheimer's disease.

In preferred embodiments, measurement of the level of transcription products of a gene coding for human MAGUIN-1 and/or human MAGUIN-2 is performed in a sample from a subject using a quantitative PCR-analysis with primer combinations to amplify said gene specific sequences from cDNA obtained by reverse transcription of RNA extracted from a sample of a subject. A Northern blot with probes specific for said gene can also be applied. It might also be preferred to measure transcription products by means of chip-based micro-array technologies. These techniques are known to those of ordinary skill in the art (see Sambrook and Russell, Molecular Cloning: A Laboratory Manual, Cold Spring Harbor Laboratory Press, Cold Spring Harbor, N.Y., 2001; Schena M., Microarray Biochip Technology, Eaton Publishing, Natick, Mass., 2000). An example of an immunoassay is the detection and measurement of enzyme activity as disclosed and described in the patent application WO 02/14543.

Furthermore, the level and/or an activity of a translation product of a gene coding for human MAGUIN-1 and/or human MAGUIN-2, and/or a fragment, or derivative, or variant of said translation product, and/or the level of activity of said translation product, and/or a fragment, or derivative, or variant thereof, can be detected using an immunoassay, an activity assay, and/or a binding assay. These assays can measure the amount of binding between said protein molecule and an anti-protein antibody by the use of enzymatic, chromodynamic, radioactive, magnetic, or luminescent labels which are attached to either the anti-protein antibody or a secondary antibody which binds the anti-protein antibody. In addition, other high affinity ligands may be used. Immunoassays which can be used include e.g. ELISAs, Western blots and other techniques known to those of ordinary skill in the art (see Harlow and Lane, Using Antibodies: A Laboratory Manual, Cold Spring Harbor Laboratory Press, Cold Spring Harbor, N.Y., 1999 and Edwards R, Immunodiagnostics: A Practical Approach, Oxford University Press, Oxford; England, 1999). All these detection techniques may also be employed in the format of microarrays, protein-arrays, antibody microarrays, tissue microarrays, electronic biochip or protein-chip based technologies (see Schena M., Microarray Biochip Technology, Eaton Publishing, Natick, Mass., 2000).

In a preferred embodiment, the level, or the activity, or both said level and said activity of (i) a transcription product of human MAGUIN-1 and/or human MAGUIN-2, and/or of (ii) a translation product of human MAGUIN-1 and/or human MAGUIN-2, and/or of (iii) a fragment, or derivative, or variant of said transcription or translation product in a series of samples taken from said subject over a period of time is compared, in order to monitor the progression of said disease. In further preferred embodiments, said subject receives a treatment prior to one or more of said sample gatherings. In yet another preferred embodiment, said level and/or activity is determined before and after said treatment of said subject.

In another aspect, the invention features a kit for diagnosing or prognosticating neurodegenerative diseases, in particular Alzheimer's disease, in a subject, or determining the propensity or predisposition of a subject to develop a neurodegenerative disease, in particular Alzheimer's disease, said kit comprising:
(a) at least one reagent which is selected from the group consisting of (i) reagents that selectively detect a transcription product of a gene coding for human MAGUIN-1 and/or human MAGUIN-2 (ii) reagents that selectively detect a translation product of a gene coding for human MAGUIN-1 and/or human MAGUIN-2; and
(b) instruction for diagnosing, or prognosticating a neurodegenerative disease, in particular Alzheimer's disease, or determining the propensity or predisposition of a subject to develop such a disease by
   detecting a level, or an activity, or both said level and said activity, of said transcription product and/or said translation product of a gene coding for human MAGUIN-1 and/or human MAGUIN-2, in a sample from said subject; and
   diagnosing or prognosticating a neurodegenerative disease, in particular Alzheimer's disease, or determining the propensity or predisposition of said subject to develop such a disease,
      wherein a varied level, or activity, or both said level and said activity, of said transcription product and/or said translation product compared to a reference value representing a known health status; or a level, or activity, or both said level and said activity, of said transcription product and/or said translation product similar or equal to a reference value representing a known disease status, indicates a diagnosis or prognosis of a neurodegenerative disease, in particular Alzheimer's disease, or an increased propensity or predisposition of developing such a disease. The kit, according to the present invention, may be particularly useful for the identification of individuals that are at risk of developing a neurodegenerative disease, in particular Alzheimer's disease. Consequently, the kit, according to the invention, may serve as a means for targeting identified individuals for early preventive measures or therapeutic intervention prior to disease onset, before irreversible damage in the course of the disease has been inflicted. Furthermore, in preferred embodiments, the kit featured in the invention is useful for monitoring a progression of a neurodegenerative disease, in particular Alzheimer's disease, in a subject, as well as monitoring success or failure of therapeutic treatment for such a disease of said subject.

In another aspect, the invention features a method of treating or preventing a neurodegenerative disease, in particular Alzheimer's disease, in a subject comprising the administration to said subject in a therapeutically or prophylactically effective amount of an agent or agents which directly or indirectly affect a level, or an activity, or both said level and said activity, of (i) a gene coding for human MAGUIN-1 and/or human MAGUIN-2, and/or (ii) a transcription product of a gene coding for human MAGUIN-1 and/or human MAGUIN-2, and/or (iii) a translation product of said gene, and/or (iv) a fragment, or derivative, or variant of (i) to (iii). Said agent may comprise a small molecule, or it may also comprise a peptide, an oligopeptide, or a polypeptide. Said peptide, oligopeptide, or polypeptide may comprise an amino acid sequence shown in SEQ ID NO.1 and/or SEQ ID NO.2, or a fragment, or derivative, or variant thereof. An agent for treating or preventing a neurodegenerative disease, in particular AD, according to the instant invention, may also consist of a nucleotide, an oligonucleotide, or a polynucleotide. Said oligonucleotide or polynucleotide may comprise a nucleotide sequence of the gene coding for human MAGUIN-1 and/or human MAGUIN-2 protein, either in sense orientation or in antisense orientation.

In preferred embodiments, the method comprises the application of per se known methods of gene therapy and/or antisense nucleic acid technology to administer said agent or agents. In general, gene therapy includes several approaches: molecular replacement of a mutated gene, addition of a new gene resulting in the synthesis of a therapeutic protein, and modulation of endogenous cellular gene expression by recombinant expression methods or by drugs. Gene-transfer techniques are described in detail (see e.g. Behr, Acc Chem Res 1993, 26: 274-278 and Mulligan, Science 1993, 260: 926-931; the contents of which are incorporated herein by reference) and include direct gene-transfer techniques such as mechanical microinjection of DNA into a cell as well as indirect techniques employing biological vectors (like recombinant viruses, especially retroviruses) or model liposomes, or techniques based on transfection with DNA coprecipitation with polycations, cell membrane pertubation by chemical (solvents, detergents, polymers, enzymes) or physical means (mechanic, osmotic, thermic, electric shocks). The postnatal gene transfer into the central nervous system has been described in detail (see e.g. Wolff, Curr Opin Neurobiol 1993, 3: 743-748).

In particular, the invention features a method of treating or preventing a neurodegenerative disease by means of antisense nucleic acid therapy, i.e. the down-regulation of an inappropriately expressed or defective gene by the introduction of antisense nucleic acids or derivatives thereof into certain critical cells (see e.g. Gillespie, DN&P 1992, 5: 389-395; Agrawal and Akhtar, Trends Biotechnol 1995, 13: 197-199; Crooke, Biotechnology 1992, 10: 882-6). Apart from hybridization strategies, the application of ribozymes, i.e. RNA molecules that act as enzymes, destroying RNA that carries the message of disease has also been described (see e.g. Barinaga, Science 1993, 262: 1512-1514). In preferred embodiments, the subject to be treated is a human, and therapeutic antisense nucleic acids or derivatives thereof are directed against human MAGUIN-1 and/or human MAGUIN-2. It is preferred that cells of the central nervous system, preferably the brain, of a subject are treated in such a way. Cell penetration can be performed by known strategies such as coupling of antisense nucleic acids and derivatives thereof to carrier particles, or the above described techniques. Strategies for administering targeted therapeutic oligodeoxynucleotides are known to those of skill in the, art (see e.g. Wickstrom, Trends Biotechnol 1992, 10: 281-287). In some cases, delivery can be performed by mere topical application. Further approaches are directed to intracellular expression of antisense RNA. In this strategy, cells are transformed ex vivo with a recombinant gene that directs the synthesis of an RNA that is complementary to a region of target nucleic acid. Therapeutical use of intracellularly expressed antisense RNA is procedurally similar to gene therapy. A recently developed method of regulating the intracellular expression of genes by the use of double-stranded RNA, known variously as RNA interference (RNAi), can be another effective approach for nucleic acid therapy (Hannon, Nature 2002, 418: 244-251).

In further preferred embodiments, the method comprises grafting donor cells into the central nervous system, preferably the brain, of said subject, or donor cells preferably treated so as to minimize or reduce graft rejection, wherein said donor cells are genetically modified by insertion of at least one transgene encoding said agent or agents. Said transgene might be carried by a viral vector, in particular a retroviral vector. The transgene can be inserted into the donor cells by a nonviral physical transfection of DNA encoding a transgene, in particular by microinjection. Insertion of the transgene can also be performed by electroporation, chemically mediated transfection, in particular calcium phosphate transfection, or liposomal mediated transfection (see Mc Celland and Pardee, Expression Genetics: Accelerated and High-Throughput Methods, Eaton Publishing, Natick, Mass., 1999).

In preferred embodiments, said agent for treating and preventing a neurodegenerative disease, in particular AD, is a therapeutic protein which can be administered to said subject, preferably a human, by a process comprising introducing subject cells into said subject, said subject cells having been treated in vitro to insert a DNA segment encoding said therapeutic protein, said subject cells expressing in vivo in said subject a therapeutically effective amount of said therapeutic protein. Said DNA segment can be inserted into said cells in vitro by a viral vector, in particular a retroviral vector.

Methods of treatment, according to the present invention, comprise the application of therapeutic cloning, transplantation, and stem cell therapy using embryonic stem cells or embryonic germ cells and neuronal adult stem cells, combined with any of the previously described cell- and gene therapeutic methods. Stem cells may be totipotent or pluripotent. They may also be organ-specific. Strategies for repairing diseased and/or damaged brain cells or tissue comprise (i) taking donor cells from an adult tissue. Nuclei of those cells are transplanted into unfertilized egg cells from which the genetic material has been removed. Embryonic stem cells are isolated from the blastocyst stage of the cells which underwent somatic cell nuclear transfer. Use of differentiation factors then leads to a directed development of the stem cells to specialized cell types, preferably neuronal cells (Lanza et al., Nature Medicine 1999, 9: 975-977), or (ii) purifying adult stem cells, isolated from the central nervous system, or from bone marrow (mesenchymal stem cells), for in vitro expansion and subsequent grafting and transplantation, or (iii) directly inducing endogenous neural stem cells to proliferate, migrate, and differentiate into functional neurons (Peterson D A, Curr. Opin. Pharmacol. 2002, 2: 34-42). Adult neural stem cells are of great potential for repairing damaged or diseased brain tissues, as the germinal centers of the adult brain are free of neuronal damage or dysfunction (Colman A, Drug Discovery World 2001, 7: 66-71).

In preferred embodiments, the subject for treatment or prevention, according to the present invention, can be a human, an experimental animal, e.g. a mouse or a rat, a domestic animal, or a non-human primate. The experimental animal can be an animal model for a neurodegenerative disorder, e.g. a transgenic mouse and/or a knock-out mouse with a neurodegenerative phenotype, in particular with an Alzheimer's-type neuropathology.

In a further aspect, the invention features a modulator of an activity, or a level, or both said activity and said level of at least one substance which is selected from the group consisting of (i) a gene coding for human MAGUIN-1 and/or human MAGUIN-2, and/or (ii) a transcription product of a gene coding for human MAGUIN-1 and/or human MAGUIN-2 and/or (iii) a translation product of a gene coding for human MAGUIN-1 and/or human MAGUIN-2, and/or (iv) a fragment, or derivative, or variant of (i) to (iii).

In an additional aspect, the invention features a pharmaceutical composition comprising said modulator and preferably a pharmaceutical carrier. Said carrier refers to a diluent, adjuvant, excipient, or vehicle with which the modulator is administered.

In a further aspect, the invention features a modulator of an activity, or a level, or both said activity and said level of at least one substance which is selected from the group consisting of (i) a gene coding for human MAGUIN-1 and/or human MAGUIN-2, and/or (ii) a transcription product of a gene coding for human MAGUIN-1 and/or human MAGUIN-2, and/or (iii) a translation product of a gene coding for human MAGUIN-1 and/or human MAGUIN-2, and/or (iv) a fragment, or derivative, or variant of (i) to (iii) for use in a pharmaceutical composition.

In another aspect, the invention provides for the use of a modulator of an activity, or a level, or both said activity and said level of at least one substance which is selected from the group consisting of (i) a gene coding for human MAGUIN-1 and/or human MAGUIN-2, and/or (ii) a transcription product of a gene coding for human MAGUIN-1 and/or human MAGUIN-2 and/or (iii) a translation product of a gene coding for human MAGUIN-1 and/or human MAGUIN-2, and/or (iv) a fragment, or derivative, or variant of (i) to (iii) for a preparation of a medicament for treating or preventing a neurodegenerative disease, in particular Alzheimer's disease.

In one aspect, the present invention also provides a kit comprising one or more containers filled with a therapeutically or prophylactically effective amount of said pharmaceutical composition.

In a further aspect, the invention features a recombinant, non-human animal comprising a non-native gene sequence coding for human MAGUIN-1 and/or human MAGUIN-2, or a fragment, or a variant, or a derivative thereof. The generation of said recombinant, non-human animal comprises (i) providing a gene targeting construct containing said gene sequence and a selectable marker sequence, and (ii) introducing said targeting construct into a stem cell of a non-human animal, and. (iii) introducing said non-human animal stem cell into a non-human embryo, and (iv) transplanting said embryo into a pseudopregnant non-human animal, and (v) allowing said embryo to develop to term, and (vi) identifying a genetically altered non-human animal whose genome comprises a modification of said gene sequence in both alleles, and (vii) breeding the genetically altered non-human animal of step (vi) to obtain a genetically altered non-human animal whose genome comprises a modification of said endogenous gene, wherein said gene is mis-expressed, or under-expressed, or over-expressed, and wherein said disruption or alteration results in said non-human animal exhibiting a predisposition to developing symptoms of neuropathology similar to a neurodegenerative disease, in particular Alzheimer's disease. Strategies and techniques for the generation and construction of such an animal are known to those of ordinary skill in the art (see e.g. Capecchi, Science 1989, 244: 1288-1292 and Hogan et al., 1994, Manipulating the Mouse Embryo: A Laboratory Manual, Cold Spring Harbor Laboratory Press, Cold Spring Harbor, N.Y. and Jackson and Abbott, Mouse Genetics and Transgenics: A Practical Approach, Oxford University Press, Oxford, England, 1999). It is preferred to make use of such a recombinant non-human animal as an animal model for investigating neurodegenerative diseases, in particular Alzheimer's disease. Such an animal may be useful for screening, testing and validating compounds, agents and modulators in the development of diagnostics and therapeutics to treat neurodegenerative diseases, in particular Alzheimer's disease.

In another aspect, the invention features an assay for screening for a modulator of neurodegenerative diseases, in particular Alzheimer's disease, or related diseases and disorders of one or more substances selected from the group consisting of (i) a gene coding for human MAGUIN-1 and/or human MAGUIN-2, and/or (ii) a transcription product of a gene coding for human MAGUIN-1 and/or human MAGUIN-2, and/or (iii) a translation product of a gene coding for human MAGUIN-1 and/or human MAGUIN-2, and/or (iv) a fragment, or derivative, or variant of (i) to (iii). This screening method comprises (a) contacting a cell with a test compound, and (b) measuring the activity, or the level, or both the activity and the level of one or more substances recited in (i) to (iv), and (c) measuring the activity, or the level, or both the activity and the level of said substances in a control cell not contacted with said test compound, and (d) comparing the levels of the substance in the cells of step (b) and (c), wherein an alteration in the activity and/or level of said substances in the contacted cells indicates that the test compound is a modulator of said diseases and disorders.

In one further aspect, the invention features a screening assay for a modulator of neurodegenerative diseases, in particular Alzheimer's disease, or related diseases and disorders of one or more substances selected from the group consisting of (i) a gene coding for human MAGUIN-1 and/or human MAGUIN-2, and/or (ii) a transcription product of a gene coding for human MAGUIN-1 and/or human MAGUIN-2, and/or (iii) a translation product of a gene coding for human MAGUIN-1 and/or human MAGUIN-2, and/or (iv) a fragment, or derivative, or variant of (i) to (iii), comprising (a) administering a test compound to a test animal which is predisposed to developing or has already developed symptoms of a neurodegenerative disease or related diseases or disorders, and (b) measuring the activity and/or level of one or more substances recited in (i) to (iv), and (c) measuring the activity and/or level of said substances in a matched control animal which is equally predisposed to developing or has already developed said symptoms and to which animal no such test compound has been administered, and (d) comparing the activity and/or level of the substance in the animals of step (b) and (c), wherein an alteration in the activity and/or level of substances in the test animal indicates that the test compound is a modulator of said diseases and disorders.

In a preferred embodiment, said test animal and/or said control animal is a recombinant, non-human animal which expresses the gene coding for human MAGUIN-1 and/or human MAGUIN-2, or a fragment, or derivative, or a variant thereof, under the control of a transcriptional regulatory element which is not the native human MAGUIN-1 and/or human MAGUIN-2 gene transcriptional control regulatory element.

In another embodiment, the present invention provides a method for producing a medicament comprising the steps of (i) identifying a modulator of neurodegenerative diseases by a method of the aforementioned screening assays and (ii) admixing the modulator with a pharmaceutical carrier. However, said modulator may also be identifiable by other types of screening assays.

In another aspect, the present invention provides for an assay for testing a compound, preferably for screening a plurality of compounds, for inhibition of binding between a ligand and human MAGUIN-1 and/or human MAGUIN-2, or a fragment, or derivative, or variant thereof. Said screening assay comprises the steps of (i) adding a liquid suspension of said human MAGUIN-1 and/or human MAGUIN-2, or a fragment, or derivative, or variant thereof, to a plurality of containers, and (ii) adding a compound or a plurality of compounds to be screened for said inhibition to said plurality of containers, and (iii) adding fluorescently labelled ligand to said containers, and (iv) incubating said human MAGUIN-1 and/or human MAGUIN-2, or said fragment, or derivative, or variant thereof, and said compound or plurality of compounds, and said fluorescently labelled ligand, and (v) measuring the amounts of fluorescence associated with said human MAGUIN-1 and/or human MAGUIN-2, or with said fragment, or derivative, or variant thereof, and (vi) determining the degree of inhibition by one or more of said compounds of binding of said ligand to said human MAGUIN-1 and/or human MAGUIN-2, or said fragment, or derivative, or variant thereof. Instead of utilizing a fluorescently labelled ligand, it might in some aspects be preferred to use any other detectable label known to the person skilled in the art, e.g. radioactive labels, and detect it accordingly. Said method may be useful for the identification of novel compounds as well as for evaluating compounds which have been improved or otherwise optimized in their ability to inhibit the binding of a ligand to a gene product of a gene coding for human MAGUIN-1 and/or human MAGUIN-2, or a fragment, or derivative, or variant thereof. One example of a fluorescent binding assay, in this case based on the use of carrier particles, is disclosed and described in patent application WO 00/52451. A further example is the competitive assay method as described in patent WO 02/01226. Preferred signal detection methods for screening assays of the instant invention are described in the following patent applications: WO 96/13744, WO 98/16814, WO 98/23942, WO 99/17086, WO 99/34195, WO 00/66985, WO 01/59436, WO 01/59416.

In one further embodiment, the present invention provides a method for producing a medicament comprising the steps of (i) identifying a compound as an inhibitor of binding between a ligand and a gene product of a gene coding for human MAGUIN-1 and/or human MAGUIN-2 by the aforementioned inhibitory binding assay and (ii) admixing the compound with a pharmaceutical carrier. However, said compound may also be identifiable by other types of screening assays.

In another aspect, the invention features an assay for testing a compound, preferably for screening a plurality of compounds to determine the degree of binding of said compounds to human MAGUIN-1 and/or human MAGUIN-2, or to a fragment, a variant, or derivative thereof. Said screening assay comprises (i) adding a liquid suspension of said human MAGUIN-1 and/or human MAGUIN-2, or a fragment, or a variant, or derivative thereof, to a plurality of containers, and (ii) adding a fluorescently labelled compound or a plurality of fluorescently labelled compounds to be screened for said binding to said plurality of containers, and (iii) incubating said human MAGUIN-1 and/or human MAGUIN-2, or said fragment, or variant, or derivative thereof, and said fluorescently labelled compound or fluorescently labelled compounds, and (iv) measuring the amounts of fluorescence associated with said human MAGUIN-1 and/or human MAGUIN-2, or with said fragment, or variant, or derivative thereof, and (v) determining the degree of binding by one or more of said compounds to said human MAGUIN-1 and/or human MAGUIN-2, or said fragment, or variant, or derivative thereof. In this type of assay it might be preferred to use a fluorescent label. However, any other type of detectable label might also be employed. Said method may be useful for the identification of novel compounds as well as for evaluating compounds which have been improved or otherwise optimized in their ability to bind to a human MAGUIN-1 and/or human MAGUIN-2 gene product, or fragment, or variant, or derivative thereof.

In one further embodiment, the present invention provides a method for producing a medicament comprising the steps of (i) identifying a compound as a binder to a gene product of a gene coding for human MAGUIN-1 and/or human MAGUIN-2 by the aforementioned binding assays and (ii) admixing the compound with a pharmaceutical carrier. However, said compound may also be identifiable by other types of screening assays.

In another embodiment, the present invention provides for a medicament obtainable by any of the methods according to the herein claimed screening assays. In one further embodiment, the instant invention provides for a medicament obtained by any of the methods according to the herein claimed screening assays.

The present invention features protein molecules shown in SEQ ID NO. 1, and SEQ ID NO. 2, or fragments, or derivatives, or variants thereof, for use as diagnostic targets for detecting a neurodegenerative disease, preferably Alzheimer's disease.

The present invention further features protein molecules shown in SEQ ID NO. 1 and SEQ ID NO. 2, or fragments, or derivatives, or variants thereof, for use as screening targets for reagents or compounds preventing, or treating, or ameliorating a neurodegenerative disease, preferably Alzheimer's disease.

The invention further features an antibody specifically immunoreactive with an immunogen, wherein said immunogen is a translation product of the human MAGUIN-1 gene shown in SEQ ID NO. 1, or a fragment, or a variant, or a derivative thereof. The immunogen may comprise immunogenic or antigenic epitopes or portions of a translation product of said gene, wherein said immunogenic or antigenic portion of a translation product is a polypeptide, and wherein said polypeptide elicits an antibody response in an animal, and wherein said polypeptide is immunospecifically bound by said antibody. Methods for generating antibodies are well known in the art (see Harlow et al., Antibodies, A Laboratory Manual, Cold Spring Harbor Laboratory Press, Cold Spring Harbor, N.Y., 1988). The term “antibody”, as employed in the present invention, encompasses all forms of antibodies known in the art, such as polyclonal, monoclonal, chimeric, recombinatorial, anti-idiotypic, humanized, or single chain antibodies, as well as fragments thereof (see Dubel and Breitling, Recombinant Antibodies, Wiley-Liss, New York, N.Y., 1999). Antibodies of the present invention are useful, for instance, in a variety of diagnostic and therapeutic methods, based on state-in-the-art techniques (see Harlow and Lane, Using Antibodies: A Laboratory Manual, Cold Spring Harbor Laboratory Press, Cold Spring Harbor, N.Y., 1999 and Edwards R., Immunodiagnostics: A Practical Approach, Oxford University Press, Oxford, England, 1999) such as enzyme-immuno assays (e.g. enzyme-linked immunosorbent assay, ELISA), radioimmuno assays, chemoluminescence-immuno assays, Western-blot, immunoprecipitation and antibody microarrays. These methods involve the detection of translation products of the human MAGUIN-1 gene.

The invention also features an antibody specifically immunoreactive with an immunogen, wherein said immunogen is a translation product of the human MAGUIN-2 gene shown in SEQ ID NO. 2, or a fragment, a variant, or a derivative thereof.

In a preferred embodiment of the present invention, said antibodies can be used for detecting the pathological state of a cell in a sample from a subject, comprising immunocytochemical staining of said cell with said antibody, wherein an altered degree of staining, or an altered staining pattern in said cell compared to a cell representing a known health status indicates a pathological state of said cell. The invention is particularly suited to detect pathological structures in the brain of a subject. It is also especially suited to detect pathological cells of the muscular system, prostate, stomach, testis, ovary, adrenal glands, mammary glands, liver, spleen, lung, trachea or placenta.

Preferably, the pathological state relates to a neurodegenerative disease, in particular to Alzheimer's disease. Immunocytochemical staining of a cell can be carried out by a number of different experimental methods well known in the art. It might be preferred, however, to apply an automated method for the detection of antibody binding, wherein the determination of the degree of staining of a cell, or the determination of the cellular or subcellular staining pattern of a cell, or the topological distribution of an antigen on the cell surface or among organelles and other subcellular structures within the cell, are carried out according to the method described in U.S. Pat. No. 6,150,173.

Other features and advantages of the invention will be apparent from the following description of figures and examples which are illustrative only and not intended to limit the remainder of the disclosure in any way.

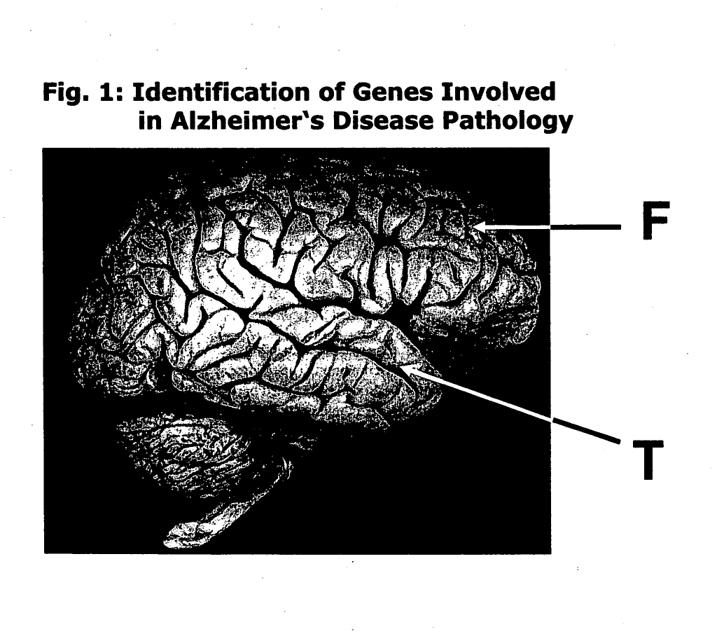

FIG. 1 depicts the brain regions with selective vulnerability to neuronal loss and degeneration in Alzheimer's disease. Primarily, neurons within the inferior temporal lobe, the entorhinal cortex, the hippocampus, and the amygdala are subject to degenerative processes in Alzheimer's disease (Terry et al., Annals of Neurology 1981, 10:184-192). These brain regions are mostly involved in the processing of learning and memory functions. In contrast, neurons within the frontal cortex, the occipital cortex, and the cerebellum remain largely intact and preserved from neurodegenerative processes in Alzheimer's disease. Brain tissues from the frontal cortex (F), the temporal cortex (T), and the hippocampus (H) of Alzheimer's disease patients and healthy, age-matched control individuals were used for the herein disclosed examples. For illustrative purposes, the image of a normal healthy brain was taken from a publication by Strange (Brain Biochemistry and Brain Disorders, Oxford University Press, Oxford, 1992, p.4).

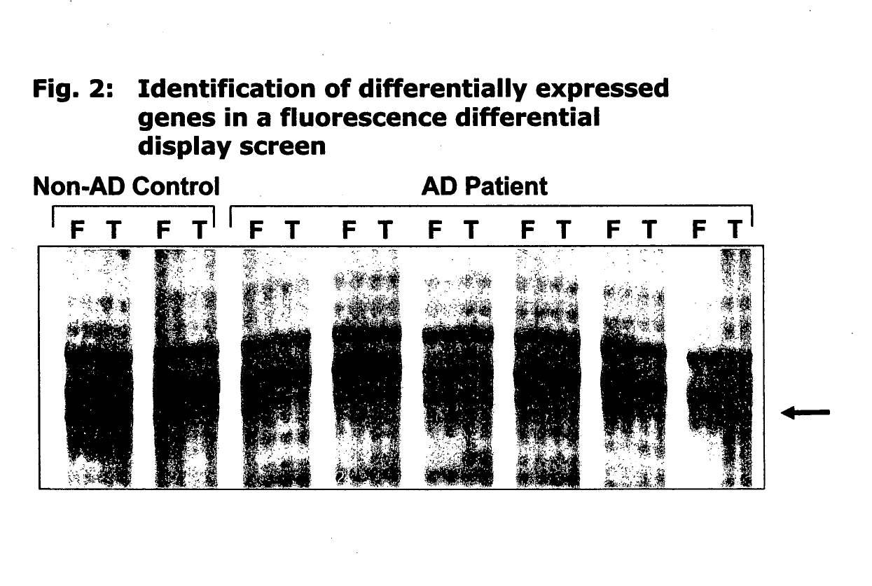

FIG. 2 discloses the initial identification of the differential expression of the human MAGUIN-1 gene in a fluorescence differential display screen. The figure shows a clipping of a large preparative fluorescent differential display gel. PCR products from the frontal cortex (F) and the temporal cortex (T) of two healthy control subjects and six Alzheimer's diseased patients were loaded in duplicate onto a denaturing polyacrylamide gel (from left to right). PCR products were obtained by amplification of the individual cDNAs with the corresponding one-base-anchor oligonucleotide and the specific Cy3 labelled random primers. The arrow indicates the lane where significant differences in intensity of the signals for human MAGUIN-1 transcript derived from frontal cortex, compared to the signals for human MAGUIN-1 transcript derived from the temporal cortex of Alzheimer's patients exist. The differential expression reflects a down-regulation of human MAGUIN RNA expression in the temporal cortex in comparison to the frontal cortex of AD patients. Comparing the signals derived from frontal cortex and temporal cortex of healthy non-AD control subjects with each other, no distinction in signal intensity, i.e. no altered expression level can be detected.

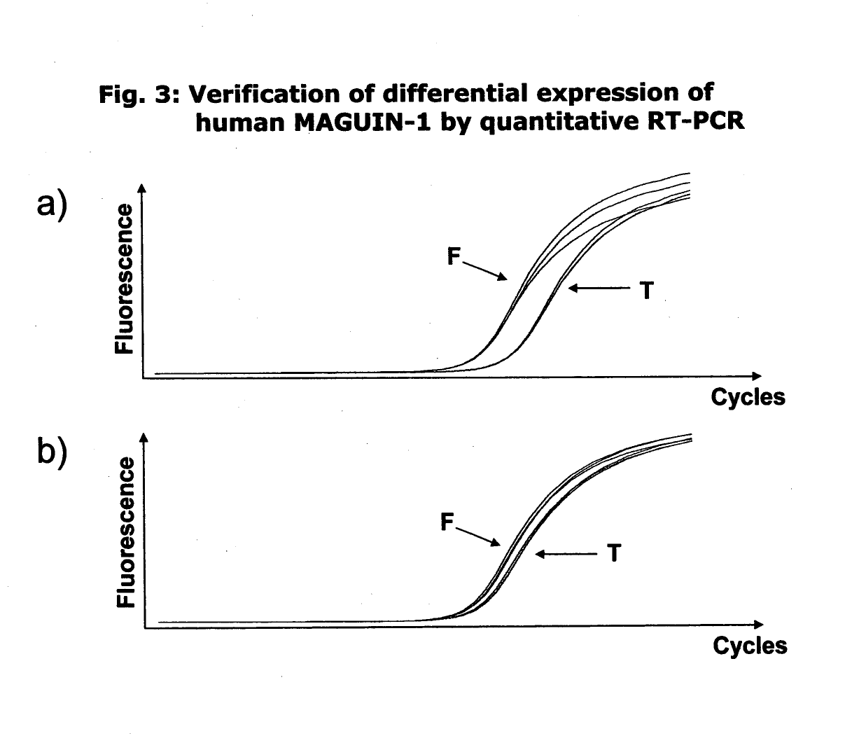

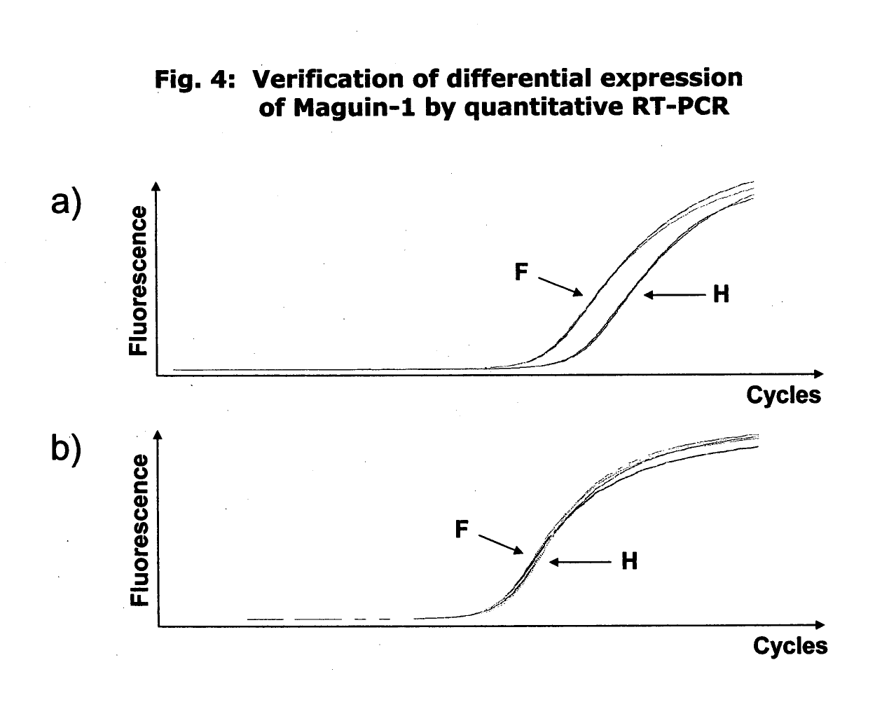

FIG. 3 and FIG. 4 illustrate the verification of the differential expression of human MAGUIN-1 in AD brain tissues by quantitative RT-PCR analysis. Quantification of RT-PCR products from RNA samples collected from the frontal cortex (F) and the temporal cortex (T) of AD patients (FIG. 3a) and samples from the frontal cortex (F) and the hippocampus (H) of AD patients (FIG. 4a) was performed by the LightCycler rapid thermal cycling technique. Likewise, samples of healthy, age-matched control individuals were compared (FIG. 3b for frontal cortex and temporal cortex, FIG. 4b for frontal cortex and hippocampus). The data were normalized to the combined average values of a set of standard genes which showed no significant differences in their gene expression levels. Said set of standard genes consisted of genes for cyclophilin B, the ribosomal protein S9, the transferrin receptor, GAPDH, and beta-actin. The figure depicts the kinetics of amplification by plotting the cycle number against the amount of amplified material as measured by its fluorescence. Note that the amplification kinetics of Maguin-1 cDNA from both, the frontal and temporal cortices of a normal control individual, and from the frontal cortex and hippocampus of a normal control individual, respectively, during the exponential phase of the reaction are juxtaposed (FIGS. 3b and 4b, arrowheads), whereas in Alzheimer's disease (FIGS. 3a and 4a, arrowheads) there is a significant separation of the corresponding curves, indicating a differential expression of Maguin-1 in the respective analyzed brain regions.

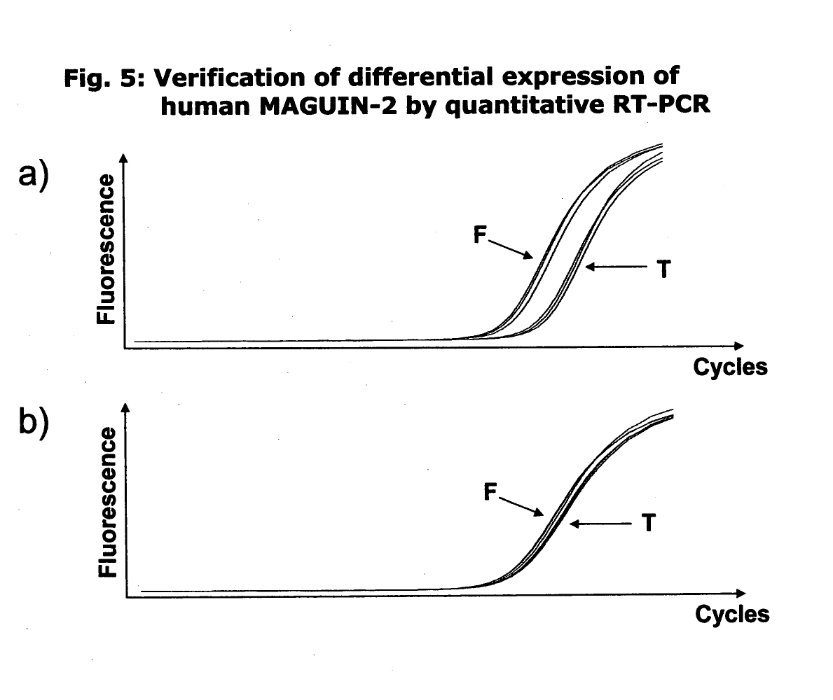

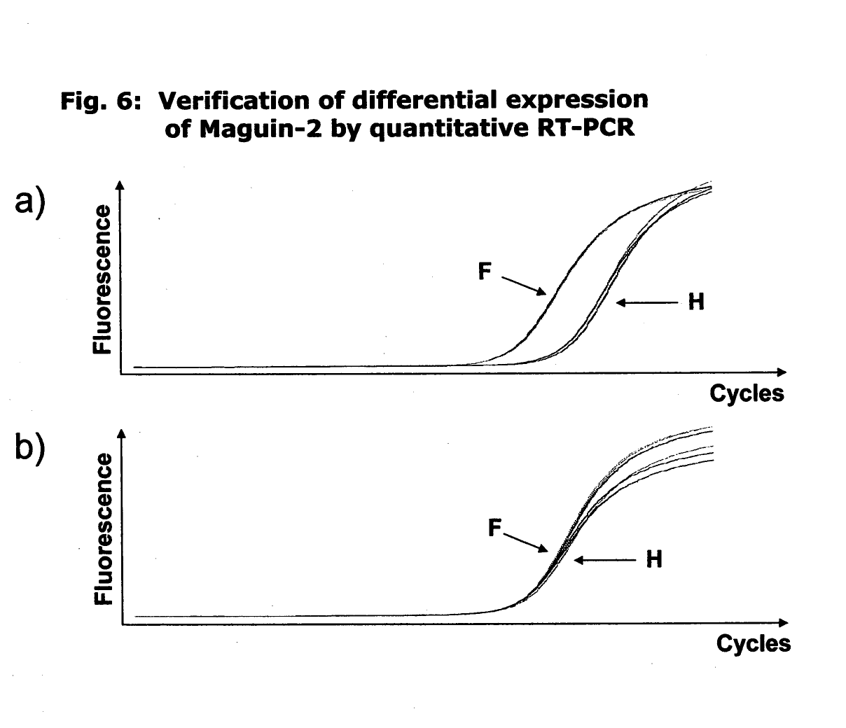

FIG. 5 and FIG. 6 illustrate the verification of the differential expression of human MAGUIN-2 in AD brain tissues by quantitative RT-PCR analysis. Quantification of RT-PCR products from RNA samples collected from the frontal cortex (F) and the temporal cortex (T) of AD patients (FIG. 5a) and samples from the frontal cortex (F) and the hippocampus (H) of AD patients (FIG. 6a) was performed by the LightCycler rapid thermal cycling technique. Likewise, samples of healthy, age-matched control individuals were compared (FIG. 5b for frontal cortex and temporal cortex, FIG. 6b for frontal cortex and hippocampus). The data were normalized to the combined average values of a set of standard genes which showed no significant differences in their gene expression levels. Said set of standard genes consisted of genes for cyclophilin B, the ribosomal protein S9, the transferrin receptor, GAPDH, and beta-actin. The figure depicts the kinetics of amplification by plotting the cycle number against the amount of amplified material as measured by its fluorescence. Note that the amplification kinetics of Maguin-2 cDNA from both, the frontal and temporal cortices of a normal control individual, and from the frontal cortex and hippocampus of a normal control individual, respectively, during the exponential phase of the reaction are juxtaposed (FIGS. 5b and 6b, arrowheads), whereas in Alzheimer's disease (FIGS. 5a and 6a, arrowheads) there is a significant separation of the corresponding curves, indicating a differential expression of Maguin-2 in the respective analyzed brain regions.

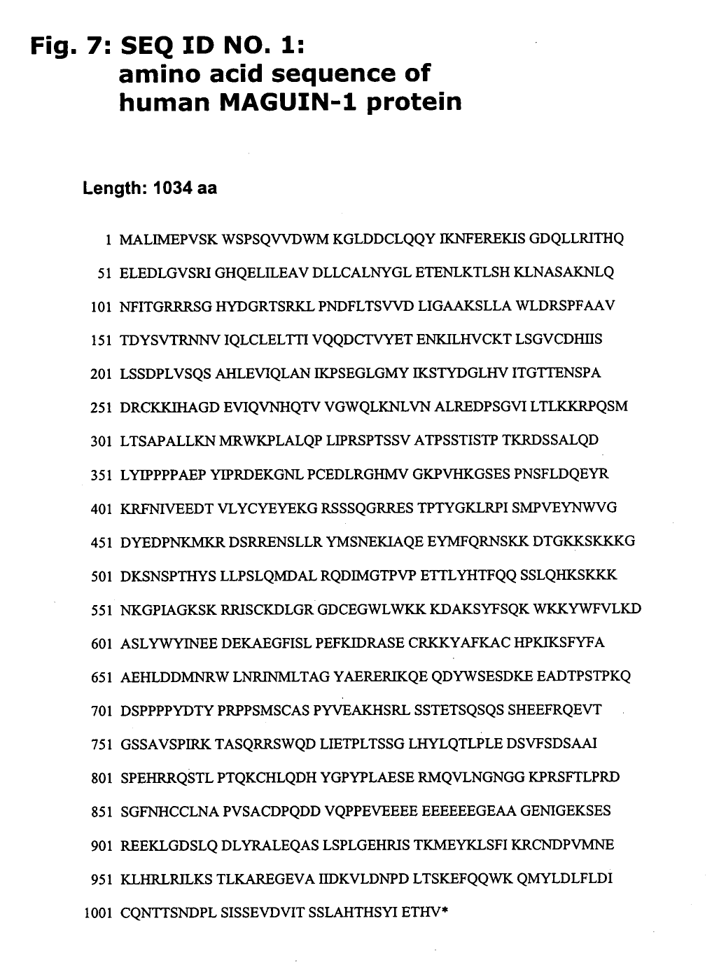

FIG. 7 discloses the amino acid sequence of the human MAGUIN-1 protein comprising 1034 amino acids; SEQ ID NO. 1. The protein harbors several distinct functional domains which are located as follows: amino acid residues 8 to 75 form the ‘Sterile Alpha Motif’ (SAM), amino acid residues 156 to 296 constitute the PDZ domain, and the Pleckstrin-homology (PH) domain consists of amino acid residues 572 to 667.

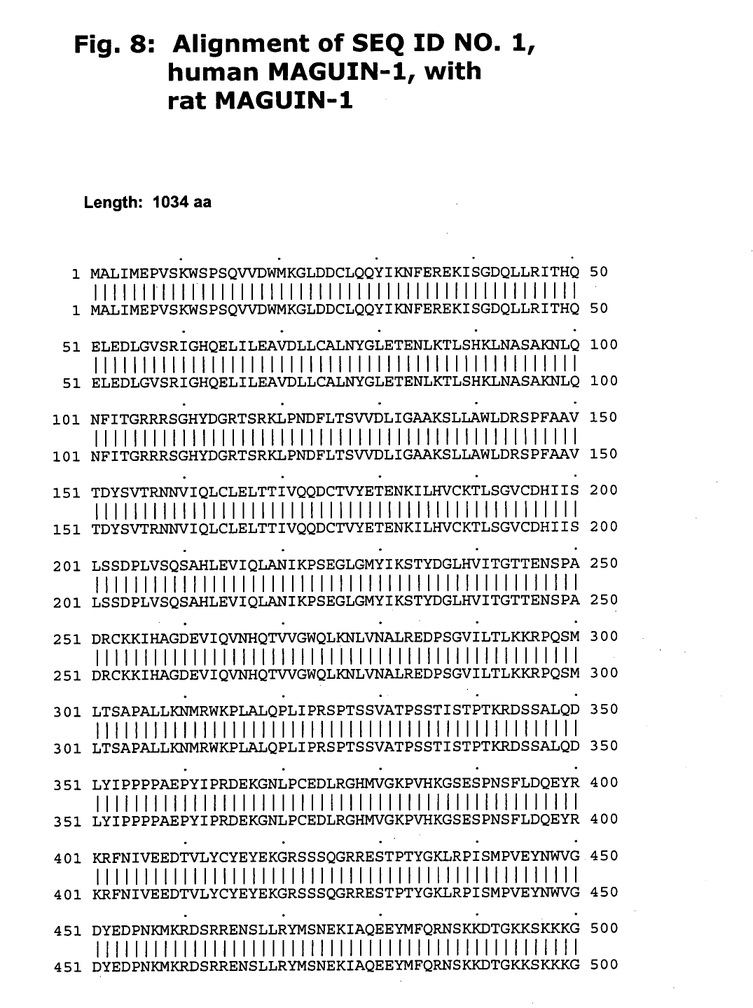

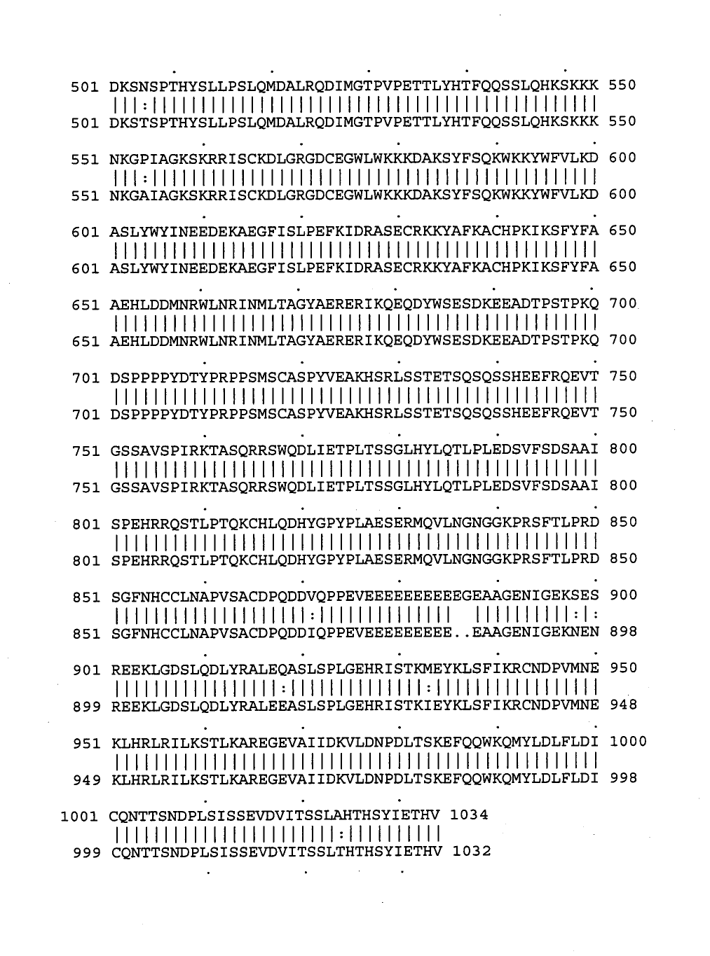

FIG. 8 shows an alignment of the amino acid sequence of SEQ ID NO.1, human MAGUIN-1, with the rat MAGUIN-1 amino acid sequence (GenBank accession number aad04568). The full length human MAGUIN-1 protein consists of 1034 amino acids (residues given in the single-letter amino acid code).

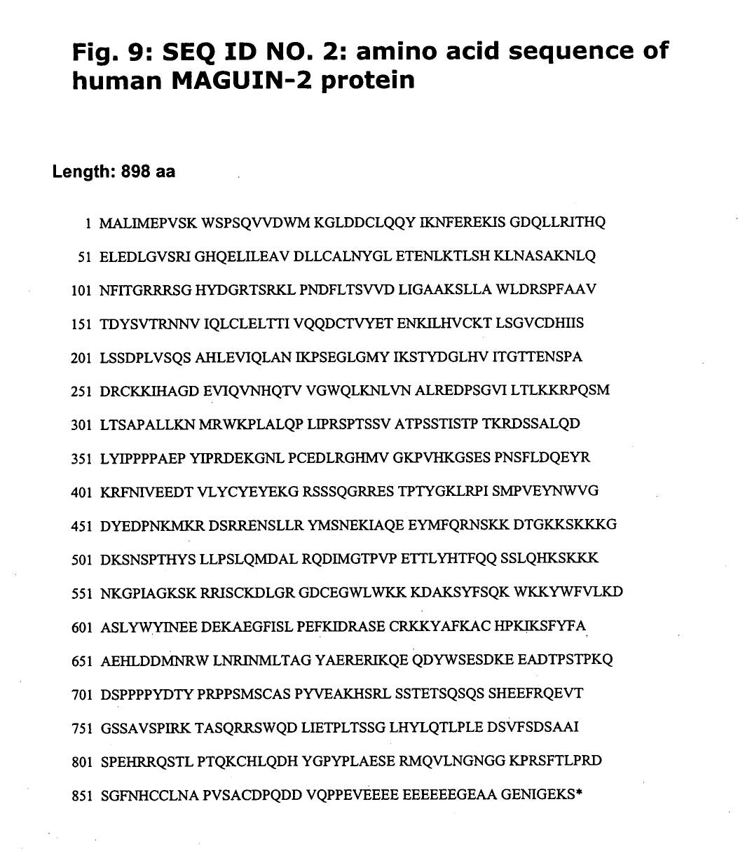

FIG. 9 discloses the amino acid sequence of the human MAGUIN-2 protein comprising 898 amino acids; SEQ ID NO. 2. The protein harbors several distinct functional domains which are located as follows: amino acid residues 8 to 75 form the ‘Sterile Alpha Motif’ (SAM), amino acid residues 156 to 296 constitute the PDZ domain, and the Pleckstrin-homology (PH) domain consists of amino acid residues 572 to 667.

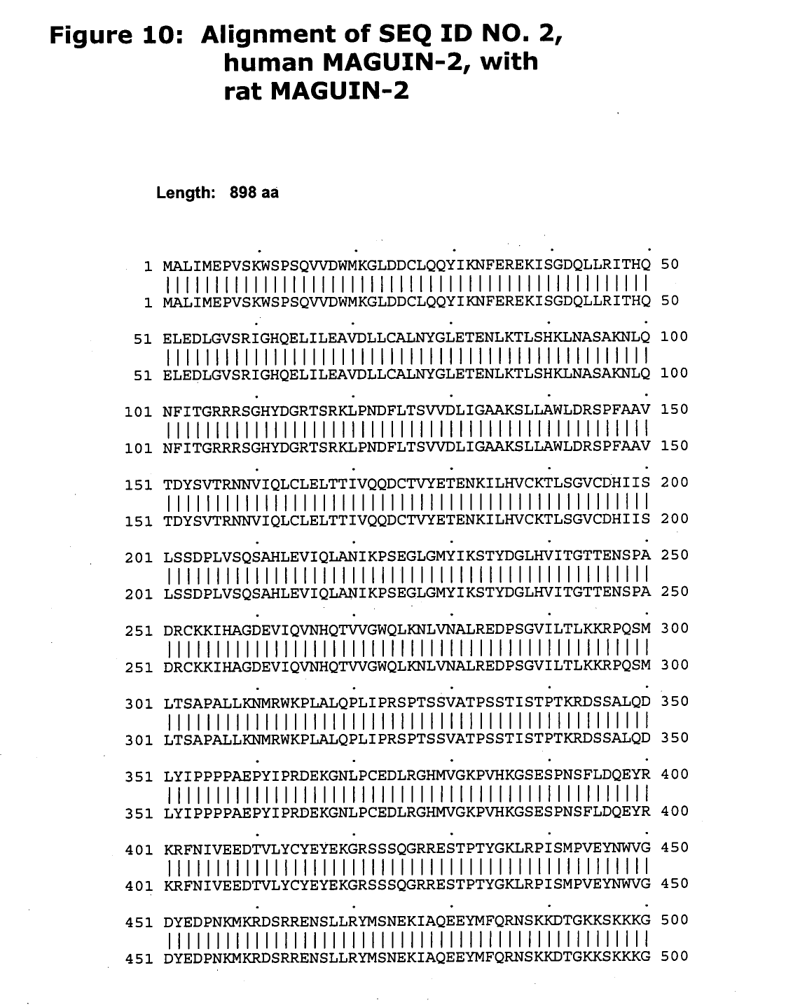

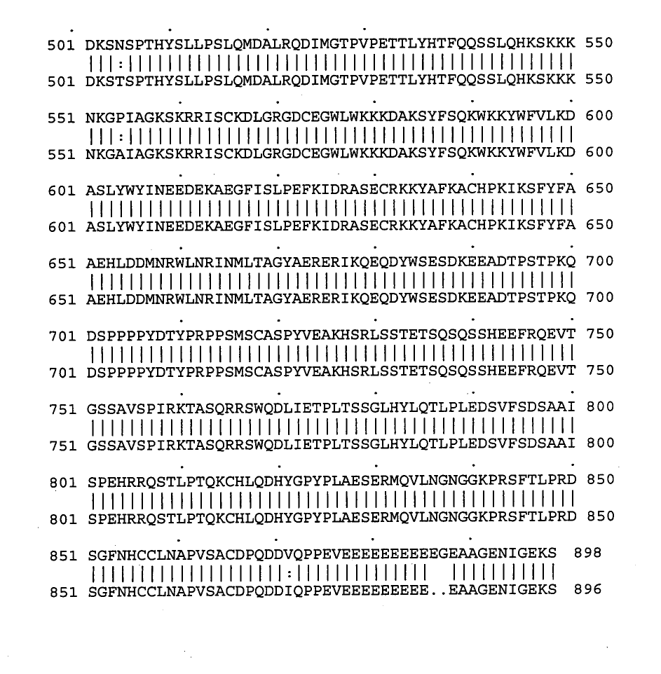

FIG. 10 shows an alignment of the amino acid sequence of SEQ ID NO. 2, human MAGUIN-2, with rat MAGUIN-2 amino acid sequence (GenBank accession number aad04567). The full length human MAGUIN-2 protein consists of 898 amino acids (residues given in the single-letter amino acid code).

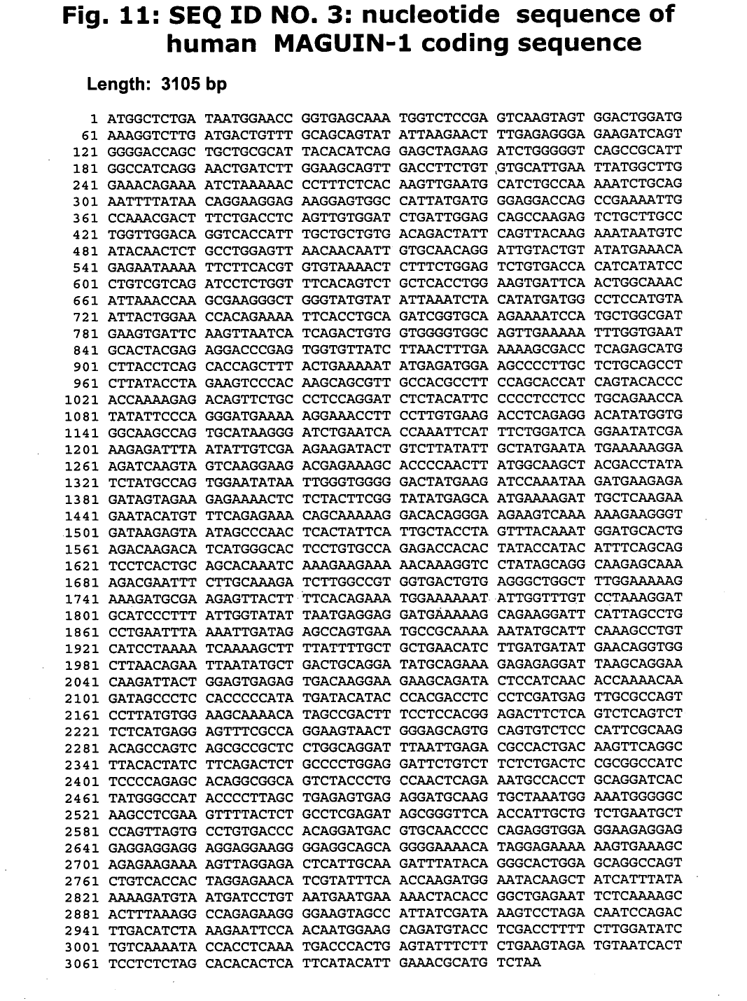

FIG. 11 represents the nucleotide sequence of SEQ ID NO. 3, the coding sequence of the human MAGUIN-1 gene, comprising 3105 nucleotides.

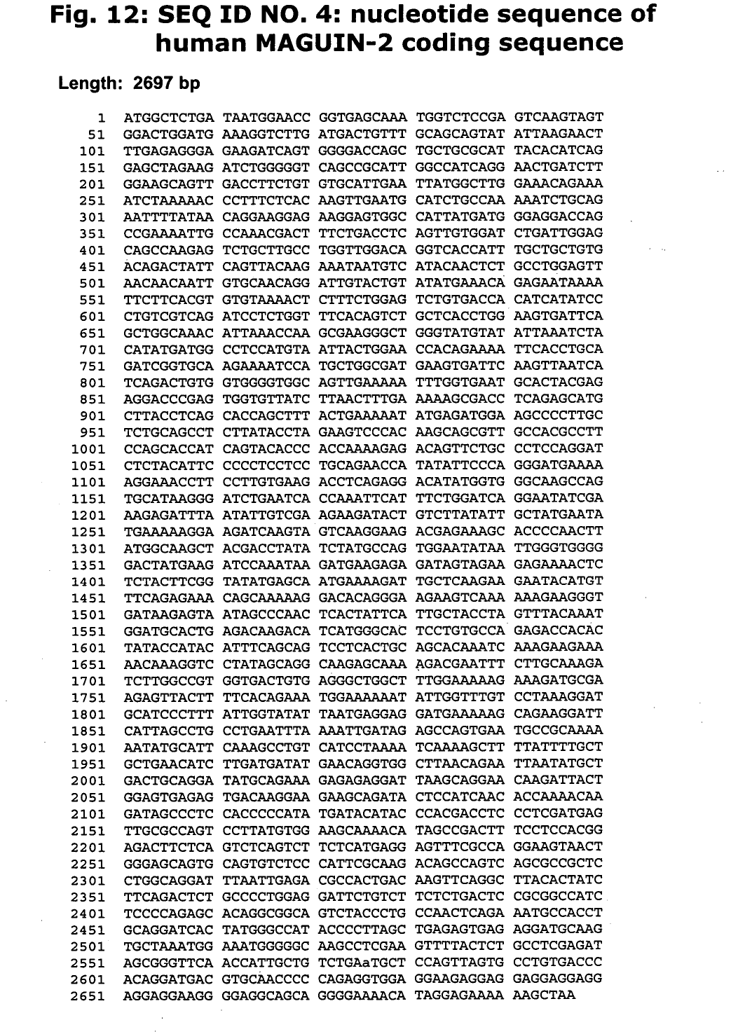

FIG. 12 represents the nucleotide sequence of SEQ ID NO. 4, the coding sequence of the human MAGUIN-2 gene, comprising 2697 nucleotides.

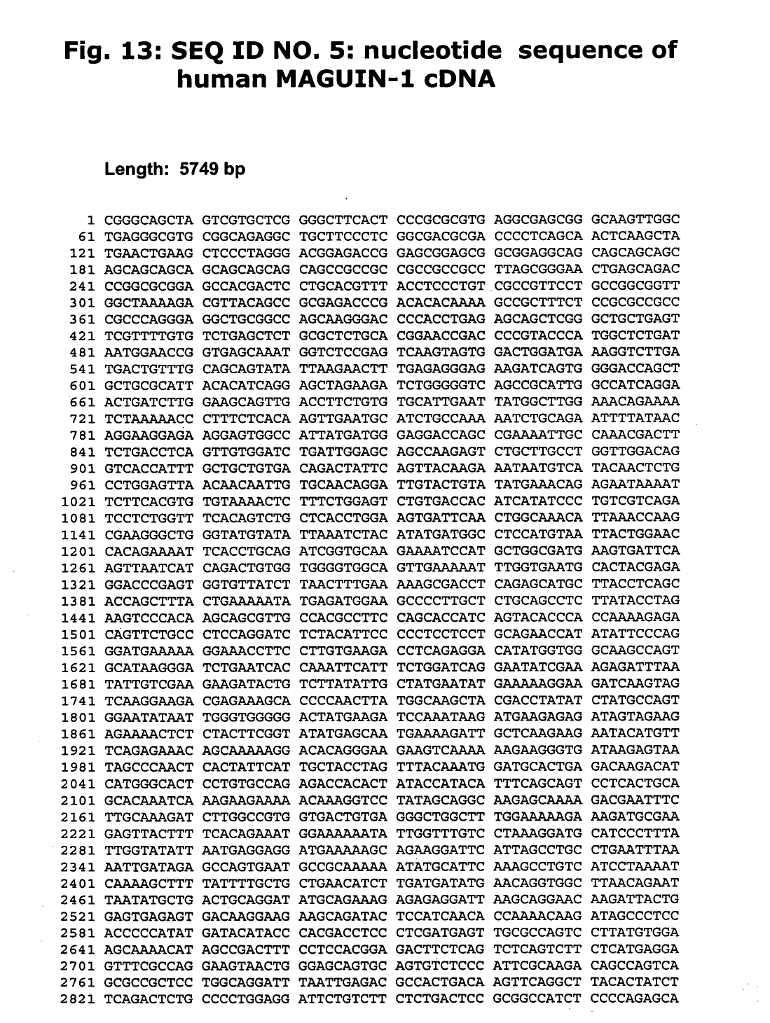

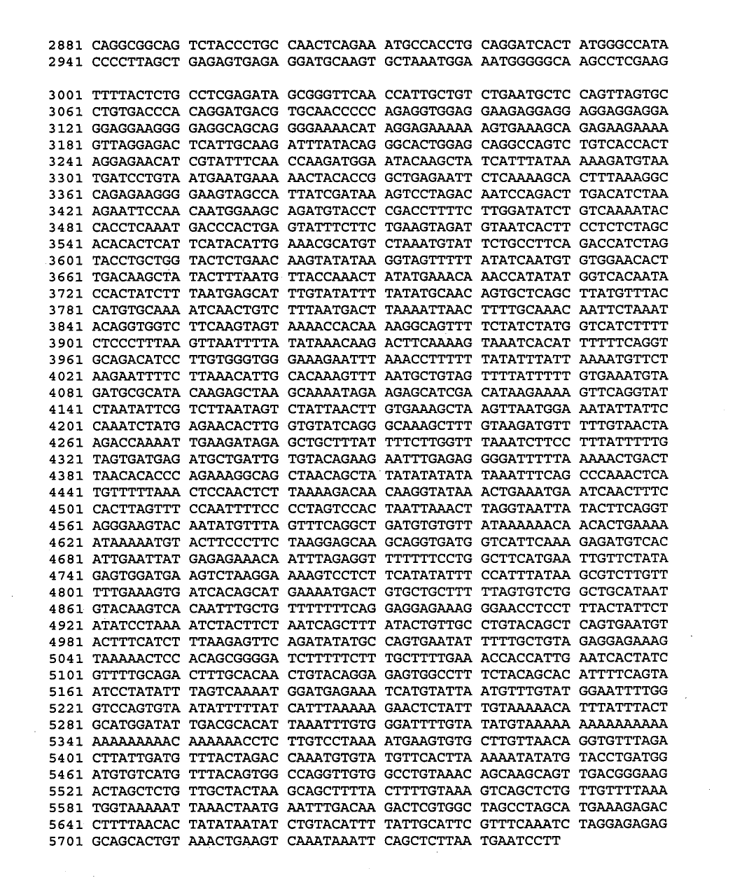

FIG. 13 shows SEQ ID NO. 5, the nucleotide sequence of the human, MAGUIN-1 cDNA, comprising 5749 nucleotides.

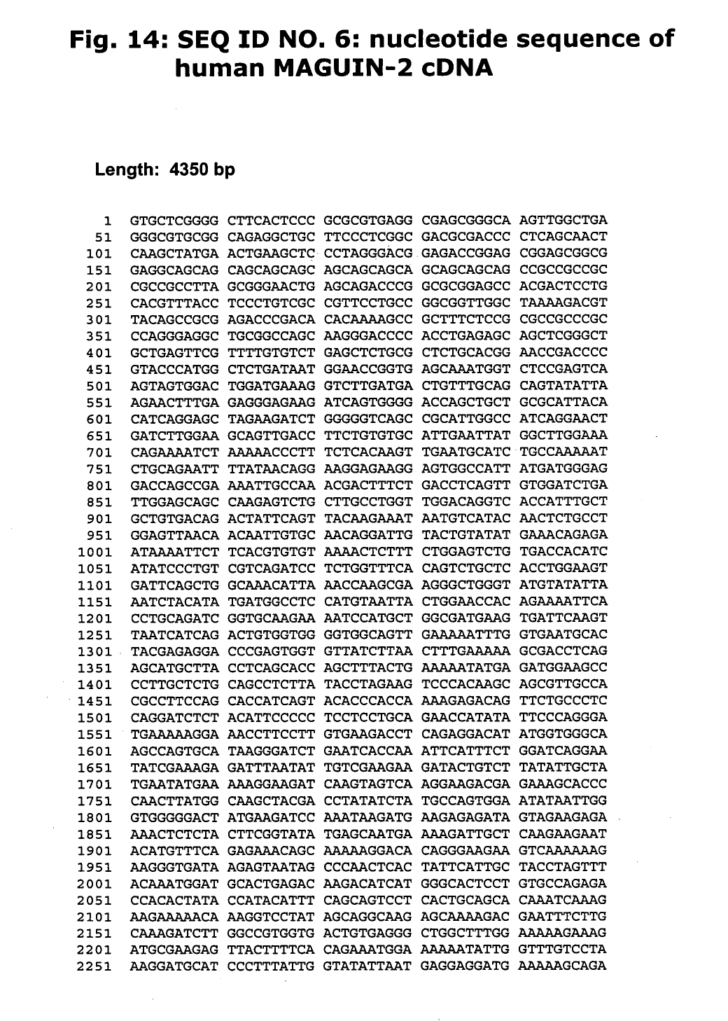

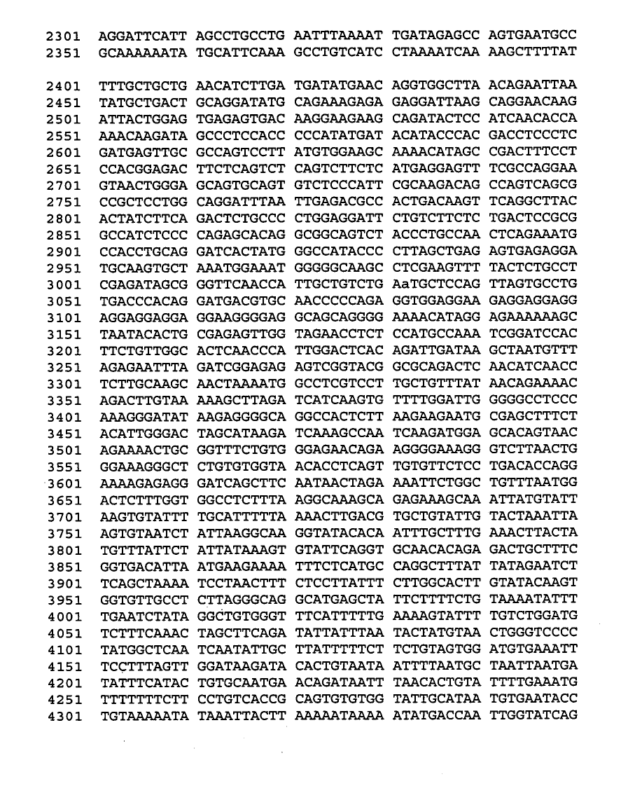

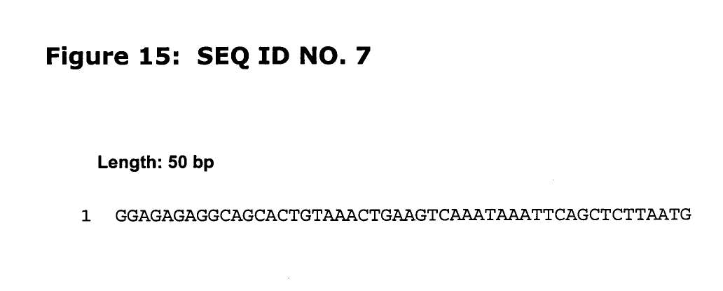

FIG. 14 shows SEQ ID NO. 6, the nucleotide sequence of the human MAGUIN-2 cDNA; comprising 4350 nucleotides.

FIG. 15 depicts SEQ ID NO. 7, the nucleotide sequence of the 50 bp MAGUIN-1 cDNA fragment, identified and obtained by fluorescence differential display and subsequent cloning.

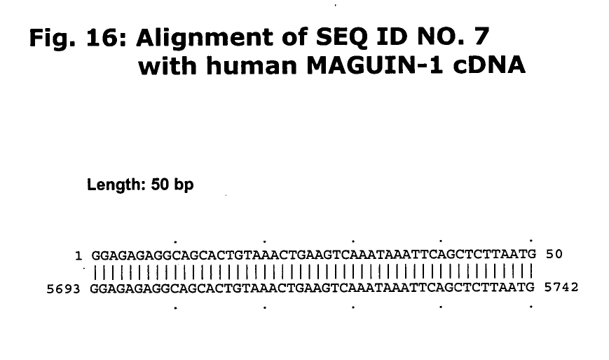

FIG. 16 outlines the sequence alignment of SEQ ID NO. 7, the 50 bp human MAGUIN-1 cDNA fragment, with the 3′UTR nucleotide sequence of SEQ ID NO. 5 (nucleotide 5693 to 5742), the nucleotide sequence of human MAGUIN-1 cDNA.

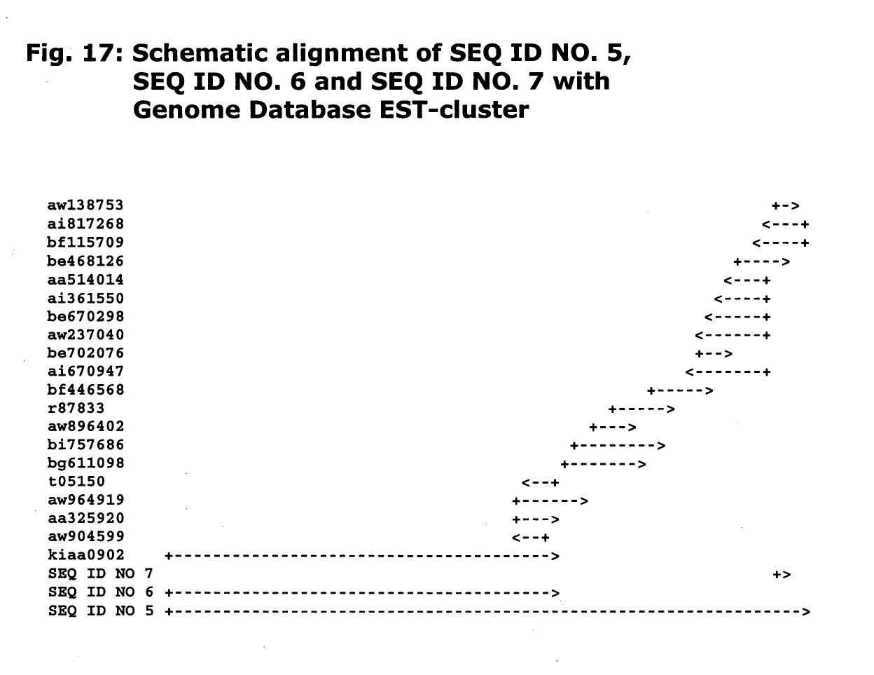

FIG. 17 charts the schematic alignment of SEQ ID NO. 7, the human MAGUIN-1 cDNA fragment, SEQ ID NO. 6, the human MAGUIN-2 cDNA sequence and the nucleotide sequence of SEQ ID NO. 5, the nucleotide sequence of human MAGUIN-1 cDNA, derived from the alignment of EST nucleotide sequences as found in the GenBank genetic sequence database. EST numbers are written on the left side, all sequences are 5′ to 3′ directed.

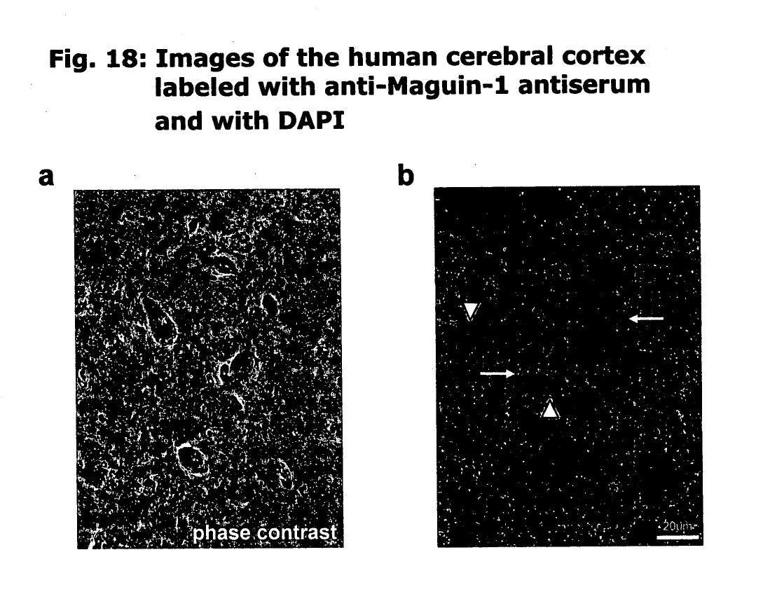

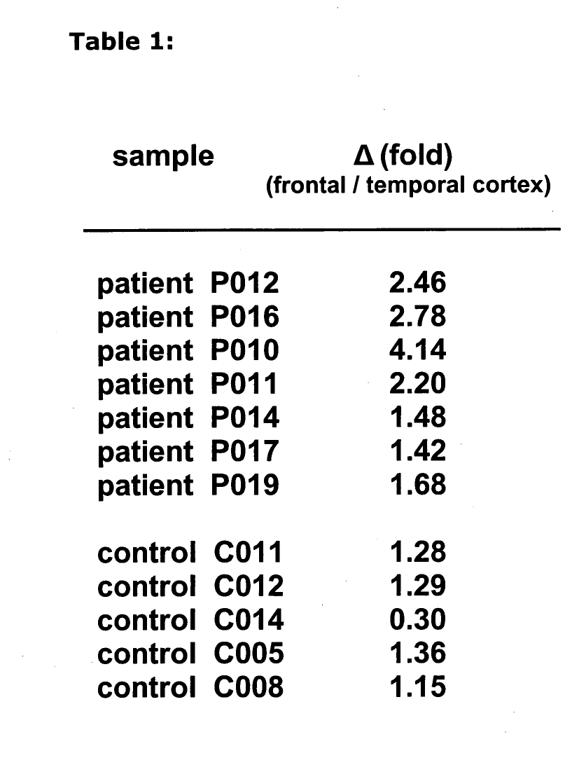

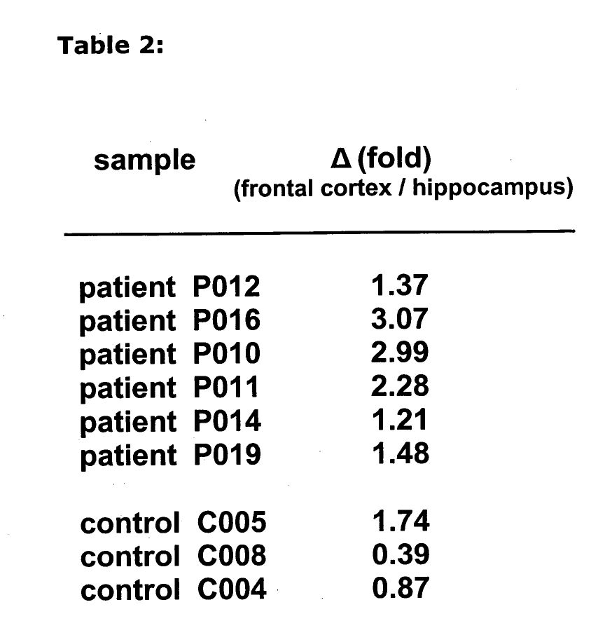

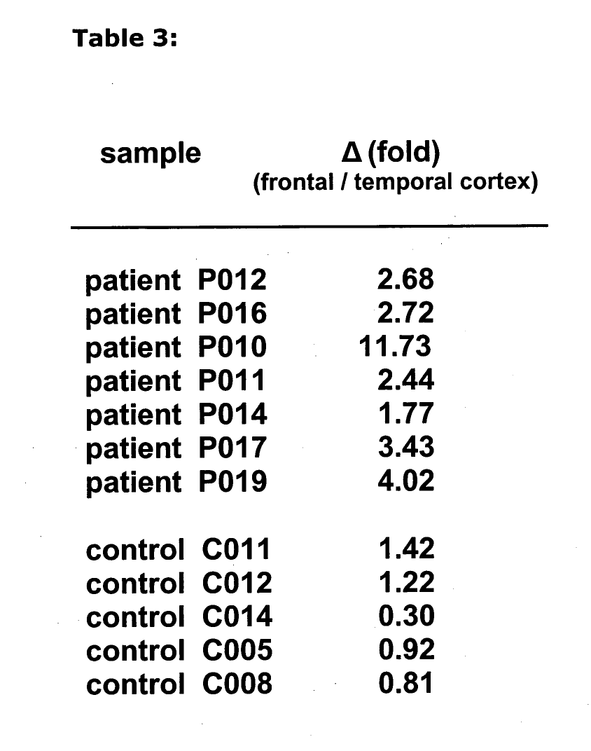

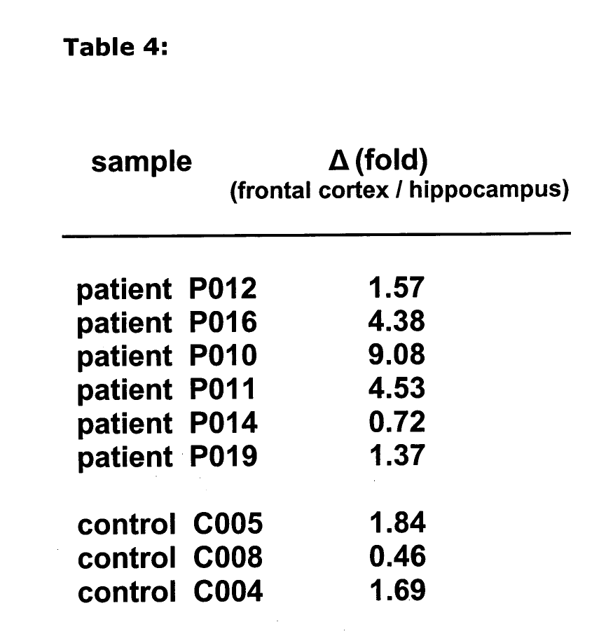

FIG. 18 depicts human cerebral cortex labeled with an affinity-purified rabbit anti-Maguin-1 antiserum raised against a peptide corresponding to amino acids 914-928 (green signals). Strong immunoreactivity of human Maguin-1 was detected in neuronal cell bodies (indicated by arrowheads) and in neurites, whereas glial cells were immuno-negative (see arrows). The same immunostaining pattern was observed by using another antiserum raised against a peptide mapping to amino acids 973-987 of Maguin-

1. Blue signals indicate nuclei stained with DAPI.

Table 1 lists the gene expression levels in the frontal cortex relative to the temporal cortex for the human MAGUIN-1 gene in seven Alzheimer's disease patients, herein identified by internal reference numbers P010, P011, P012, P014, P016, P017, P019 (1.42 to 4.14 fold) and five healthy, age-matched control individuals, herein identified by internal reference numbers C005, C008, C011, C012, C014 (0.30 to 1.36 fold). The values shown are reciprocal values according to the formula described herein.

Table 2 lists the gene expression levels in the frontal cortex relative to the hippocampus for the human MAGUIN-1 gene in six Alzheimer's disease patients, herein identified by internal reference numbers P010, P011, P012, P014, P016, P019 (1.21 to 3.07 fold) and three healthy, age-matched control individuals, herein identified by internal reference numbers C004, C005, C008 (0.39 to 1.74 fold). The values shown are reciprocal values according to the formula described herein.

Table 3 lists the gene expression levels in the frontal cortex relative to the temporal cortex for the human MAGUIN-2 gene in seven Alzheimer's disease patients, herein identified by internal reference numbers P010, P011, P012, P014, P016, P017, P019 (1.77 to 11.73 fold) and five healthy, age-matched control individuals, herein identified by internal reference numbers C005, C008, C011, C012, C014 (0.30 to 1.42 fold). The values shown are reciprocal values according to the formula described herein.

Table 4 lists the gene expression levels in the frontal cortex relative to the hippocampus for the human MAGUIN-2 gene in six Alzheimer's disease patients, herein identified by internal reference numbers P010, P011, P012, P014, P016, P019 (0.72 to 9.08 fold) and three healthy, age-matched control individuals, herein identified by internal reference numbers C004, C005, C008 (0.46 to 1.69 fold). The values shown are reciprocal values according to the formula described herein.

### EXAMPLE I

(i) Brain Tissue Dissection from Patients with Alzheimer's Disease:

Brain tissues from Alzheimer's disease patients and age-matched control subjects were collected within 6 hours post-mortem and immediately frozen on dry ice. Sample sections from each tissue were fixed in paraformaldehyde for histopathological confirmation of the diagnosis. Brain areas for differential expression analysis were identified (see FIG. 1) and stored at −80° C. until RNA extractions were performed.

(ii) Isolation of Total RNA:

Total RNA was extracted from post-mortem brain tissue by using the RNeasy kit (Qiagen) according to the manufacturer's protocol. The accurate RNA concentration and the RNA quality were determined with the DNA LabChip system using the Agilent 2100 Bioanalyzer (Agilent Technologies). For additional quality testing of the prepared RNA, i.e. exclusion of partial degradation and testing for DNA contamination, specifically designed intronic GAPDH oligonucleotides and genomic DNA as reference control were utilised to generate a melting curve with the LightCycler technology as described in the manufacturer's protocol (Roche).

(iii) cDNA Synthesis and Identification of Differentially Expressed Genes by Fluorescence Differential Display (FDD):

In order to identify changes in gene expression in different tissues we employed a modified and improved differential display (DD) screening method. The original DD screening method is known to those skilled in the art (Liang and Pardee, Science 1995, 267:1186-7). This technique compares two populations of RNA and provides clones of genes that are expressed in one population but not in the other. Several samples can be analyzed simultaneously and both up- and down-regulated genes can be identified in the same experiment. By adjusting and refining several steps in the DD method as well as modifying technical parameters, e.g. increasing redundancy, evaluating optimized reagents and conditions for reverse transcription of total RNA, optimizing polymerase chain reactions (PCR) and separation of the products thereof, a technique was developed which allows for highly reproducible and sensitive results. The applied and improved DD technique was described in detail by von der Kammer et al. (Nucleic Acids Research 1999, 27: 2211-2218). A set of 64 specifically designed random primers were developed (standard set) to achieve a statistically comprehensive analysis of all possible RNA species. Further, the method was modified to generate a preparative DD slab-gel technique, based on the use of fluorescently labelled primers. In the present invention, RNA populations from carefully selected post-mortem brain tissues (frontal and temporal cortex) of Alzheimer's disease patients and age-matched control subjects were compared.

As starting material for the DD analysis we used total RNA, extracted as described above (ii). Equal amounts of 0.05 μg RNA each were transcribed into cDNA in 20 μl reactions containing 0.5 mM each dNTP, 1 μl Sensiscript™ Reverse Transcriptase and 1× RT buffer (Qiagen), 10 U RNase inhibitor (Qiagen) and 1 μM of either one-base-anchor oligonucleotides HT₁₁A, HT₁₁G or HT₁₁C (Liang et al., Nucleic Acids Research 1994, 22: 5763-5764; Zhao et al., Biotechniques 1995, 18: 842-850). Reverse transcription was performed for 60 min at 37° C. with a final denaturation step at 93° C. for 5 min. 2 μl of the obtained cDNA each was subjected to a polymerase chain reaction (PCR) employing the corresponding one-base-anchor oligonucleotide (1 μM) along with either one of the Cy3 labelled random DD primers (1 μM), 1× GeneAmp PCR buffer (Applied Biosystems,), 1.5 mM MgCl₂ (Applied Biosystems), 2 μM dNTP-Mix (dATP, dGTP, dCTP, dTTP Amersham Pharmacia Biotech), 5% DMSO (Sigma), 1 U AmpliTaq DNA Polymerase (Applied Biosystems) in a 20 μl final volume. PCR conditions were set as follows: one round at 94° C. for 30 sec for denaturing, cooling 1° C./sec down to 40° C., 40° C. for 4 min for low-stringency annealing of primer, heating 1° C./sec up to 72° C., 72° C. for 1 min for extension. This round was followed by 39 high-stringency cycles: 94° C. for 30 sec, cooling 1° C./sec down to 60° C., 60° C. for 2 min, heating 1° C./sec up to 72° C., 72° C. for 1 min. One final step at 72° C. for 5 min was added to the last cycle (PCR cycler: Multi Cycler PTC 200, MJ Research). 8 μl DNA loading buffer were added to the 20 μl PCR product preparation, denatured for 5 min and kept on ice until loading onto a gel. 3.5 μl each were separated on 0.4 mm thick, 6%-polyacrylamide (Long Ranger)/7 M urea sequencing gels in a slab-gel system (Hitachi Genetic Systems) at 2000 V, 60W, 30 mA, for 1 h 40 min. Following completion of the electrophoresis, gels were scanned with a FMBIO II fluorescence-scanner (Hitachi Genetic Systems), using the appropriate FMBIO II Analysis 8.0 software. A full-scale picture was printed, differentially expressed bands marked, excised from the gel, transferred into 1.5 ml containers, overlayed with 200 μl sterile water and kept at −20° C. until extraction.

Elution and reamplification of differential display products: The differential bands were extracted from the gel by boiling in 200 μl H₂O for 10 min, cooling down on ice and precipitation from the supernatant fluids by using ethanol (Merck) and glycogen/sodium acetate (Merck) at −20° C. over night, and subsequent centrifugation at 13.000 rpm for 25 min at 4° C. Pellets were washed twice in ice-cold ethanol (80%), resuspended in 10 mM Tris pH 8.3 (Merck) and dialysed against 10% glycerol (Merck) for 1 h at room temperature on a 0.025 μm VSWP membrane (Millipore). The obtained preparations were used as templates for reamplification by 15 high-stringency cycles in 25-μl PCR mixtures containing the corresponding primer pairs as used for the differential display PCR (see above) under identical conditions, with the exception of the initial round at 94° C. for 5 min, followed by 15 cycles of: 94° C. for 45 sec, 60° C. for 45 sec, ramp 1° C./sec to 70° C. for 45 sec, and one final step at 72° C. for 5 min.

Cloning and sequencing of differential display products: Re-amplified cDNAs were analyzed with the DNA LabChip system (Agilent 2100 Bioanalyzer, Agilent Technologies) and were ligated into the pCR-Blunt II-TOPO vector and transformed into E.coli Top10F′ cells (Zero Blunt TOPO PCR Cloning Kit, Invitrogen) according to the manufacturer's instructions. Cloned cDNA fragments were sequenced by commercially available sequencing facilities. The results of one such fluorescence differential display experiment for the human MAGUIN-1 gene are shown in FIG. 2.

(iv) Confirmation of Differential Expression by Quantitative RT-PCR:

Positive corroboration of differential expression of the human MAGUIN-1 gene and human MAGUIN-2 gene was performed using the LightCycler technology (Roche). This technique features rapid thermal cyling for the polymerase chain reaction as well as real-time measurement of fluorescent signals during amplification and therefore allows for highly accurate quantification of RT-PCR products by using a kinetic, rather than an endpoint approach. The ratios of human MAGUIN-1 and human MAGUIN-2 cDNA each from the temporal cortex and frontal cortex, and from the hippocampus and frontal cortex, respectively, were determined (relative quantification).

First, a standard curve was generated to determine the efficiency of the PCR with specific primers for human MAGUIN-1:

5′-CAGCAAGCAGTTGACGGGA-3′
and
5′-GCCACGAGTCTTGTCAAATTCA-3′

and human MAGUIN-2:

5′-GGGCCTCCCAAAGGGATAT-3′
and
5′-CCCAATGTAGAAAGCTCGCATT-3′.

PCR amplification (95° C. and 1 sec, 56° C. and 5 sec, and 72° C. and 5 sec) was performed in a volume of 20 μl containing Lightcycler-FastStart DNA Master SYBR Green I mix (contains FastStart Taq DNA polymerase, reaction buffer, dNTP mix with dUTP instead of dTTP, SYBR Green I dye, and 1 mM MgCl₂; Roche), 0.5 μM primers, 2 μl of a cDNA dilution series (final concentration of 40, 20, 10, 5, 1 and 0.5 ng human total brain cDNA; Clontech) and depending on the primers used, additional 3 mM MgCl₂. Melting curve analysis revealed a single peak at approximately 80° C. and 80.6° C. with no visible primer dimers. Quality and size of the PCR product were determined with the DNA LabChip system (Agilent 2100 Bioanalyzer, Agilent Technologies). A single peak at the expected size of 121 bp for human MAGUIN-1 and 67 bp for human MAGUIN-2 was observed in the electropherogram of the sample.

In an analogous manner, the PCR protocol was applied to determine the PCR efficiency of a set of reference genes which were selected as a reference standard for quantification. In the present invention, the mean value of five such reference genes was determined: (1) cyclophilin B, using the specific primers 5′-ACTGAAGCACTACGGGCCTG-3′ and 5′-AGCCGTTGGTGTCTTTGCC-3′ except for MgCl₂ (an additional 1 mM was added instead of 3 mM). Melting curve analysis revealed a single peak at approximately 87° C. with no visible primer dimers. Agarose gel analysis of the PCR product showed one single band of the expected size (62 bp). (2) Ribosomal protein S9 (RPS9), using the specific primers 5′-GGTCAAATTTACCCTGGCCA-3′ and 5′-TCTCATCAAGCGTCAGCAGTTC-3′ (exception: additional 1 mM MgCl₂ was added instead of 3 mM). Melting curve analysis revealed a single peak at approximately 85° C. with no visible primer dimers. Agarose gel analysis of the PCR product showed one single band with the expected size (62 bp). (3) beta-actin, using the specific primers 5′-TGGAACGGTGAAGGTGACA-3′ and 5′-GGCAAGGGACTTCCTGTAA-3′. Melting curve analysis revealed a single peak at approximately 87° C. with no visible primer dimers. Agarose gel analysis of the PCR product showed one single band with the expected size (142 bp). (4) GAPDH, using the specific primers 5′-CGTCATGGGTGTGAACCATG-3′ and 5′-GCTAAGCAGTTGGTGGTGCAG-3′. Melting curve analysis revealed a single peak at approximately 83° C. with no visible primer dimers. Agarose gel analysis of the PCR product showed one single band with the expected size (81 bp). (5) Transferrin receptor TRR, using the specific primers 5′-GTCGCTGGTCAGTTCGTGATT-3′ and 5′-AGCAGTTGGCTGTTGTACCTCTC-3′. Melting curve analysis revealed a single peak at approximately 83° C. with no visible primer dimers. Agarose gel analysis of the PCR product showed one single band with the expected size (80 bp).

For calculation of the values, first the logarithm of the cDNA concentration was plotted against the threshold cycle number Cₜ for human MAGUIN-1 and human MAGUIN-2, respectively, and the five reference standard genes. The slopes and the intercepts of the standard curves (i.e. linear regressions) were calculated for all genes. In a second step, cDNAs from temporal cortex and frontal cortex, and from hippocampus and frontal cortex, for human MAGUIN-1 and human MAGUIN-2, respectively, were analyzed in parallel and normalized to cyclophilin B. The Cₜ values were measured and converted to ng total brain cDNA using the corresponding standard curves:
10\hat{0}((Cₜ value−intercept)/slope ) [ng total brain cDNA]

The values for temporal cortex and frontal cortex of human MAGUIN-1 and human MAGUIN-2 cDNAs, and the values for hippocampus and frontal cortex of human MAGUIN-1 and human MAGUIN-2 cDNAs, respectively, were normalized to cyclophilin B, and the ratios were calculated using the following formulas:

$$Ratio = \frac{\begin{matrix}human MAGUIN \text{-} 1 ( MAGUIN \text{-} 2 ) temporal [ ng ] / \\ cyclophilin B temporal [ ng ]\end{matrix}}{\begin{matrix}human MAGUIN \text{-} 1 ( MAGUIN \text{-} 2 ) frontal [ ng ] / \\ cyclophilin B frontal [ ng ]\end{matrix}}$$

$$Ratio = \frac{\begin{matrix}human MAGUIN \text{-} 1 ( MAGUIN \text{-} 2 ) hippocampus [ ng ] / \\ cyclophilin B hippocampus [ ng ]\end{matrix}}{\begin{matrix}human MAGUIN \text{-} 1 ( MAGUIN \text{-} 2 ) frontal [ ng ] / \\ cyclophilin B frontal [ ng ]\end{matrix}}$$

In a third step, the set of reference standard genes was analyzed in parallel to determine the mean average value of the temporal to frontal ratios, and of the hippocampal to frontal ratios, respectively, of expression levels of the reference standard genes for each individual brain sample. As cyclophilin B was analyzed in step 2 and step 3, and the ratio from one gene to another gene remained constant in different runs, it was possible to normalize the values for human MAGUIN-1 and human MAGUIN-2 to the mean average value of the set of reference standard genes instead of normalizing to one single gene alone. The calculation was performed by dividing the ratio shown above by the deviation of cyclophilin B from the mean value of all housekeeping genes. The results of such quantitative RT-PCR analysis for the human MAGUIN-1 gene are shown in FIG. 3 and 4 and for the human MAGUIN-2 gene in FIG. 5 and 6.

(v) Sequence Analysis

Searching the EST database of the GenBank database for sequence similarities to the identified differentially expressed human cDNA fragment, SEQ ID NO. 7, as stated in the present invention, it was found that SEQ ID NO. 7 was identical to portions of the human EST sequences ai817268 and bf115709, (shown in FIG. 17). These human ESTs showed high homology to rat (Rattus norvegicus) MAGUIN-1. Aligning human ESTs in addition to the identified expressed SEQ ID NO. 7, a complete EST cluster representing the human MAGUIN-1 cDNA, SEQ ID NO. 5, was constructed. The amino acid sequence of a large open reading frame with the potential to encode a protein of 1034 amino acid residues was deduced (SEQ ID NO. 1). The human MAGUIN-1 protein, as denoted herein, is highly homologous to the rat MAGUIN-1 protein. In addition, it encodes a protein (SEQ ID NO. 1) harboring a number of structurally and functionally important domains. One SAM, one PDZ, and one PH domain are located from amino acid residues 8 to 75 (SAM), amino acid residues 156 to 296 (PDZ), and amino acid residues 572 to 667 (PH) (refer to FIG. 7).

(vi) Immunohistochemistry:

For immunofluorescence staining of Maguin-1 in human brain, frozen sections were prepared from post-mortem pre-central gyrus of a donor person (Cryostat Leica CM3050S) and fixed in acetone for 10 min. After washing in PBS, the sections were pre-incubated with blocking buffer (10% normal goat serum, 0.2% Triton X-100 in PBS) for 30 min, and then incubated with affinity-purified rabbit anti-Maguin-1 antisera (1:20-1:50 diluted in blocking buffer, custom-made by Davids Biotechnologie, Regensburg) overnight at 4° C. After rinsing three times in 0.2% Triton X-100/PBS, the sections were incubated with FITC-conjugated goat anti-rabbit IgG (1:150 diluted in 1% BSA/PBS) for 2 hours at room temperature, and then again washed in PBS. Staining of the nuclei was performed by incubation of the sections with 5 μM DAPI in PBS for 3 min (blue signal). In order to block the autofluoresence of lipofuscin in human brain, the sections were treated with 1% Sudan Black B in 70% ethanol for 2-10 min at room temperature, sequentially dipped in 70% ethanol, destined water and PBS. The sections were coverslipped by ‘Vectrashield mounting medium’ (Vector Laboratories, Burlingame, Calif.) and observed under an inverted microscope (IX81, Olympus Optical). The digital images were captured with the appropriate software (AnalySiS, Olympus Optical).

1. A method of diagnosing or prognosticating a neurodegenerative disease in a subject, or determining whether a subject is at increased risk of developing said disease, comprising:
determining a level and/or an activity of
(i) a transcription product of a gene coding for human MAGUIN-1 and/or human MAGUIN-2, and/or
(ii) a translation product of a gene coding for human MAGUIN-1 and/or human MAGUIN-2, and/or
(iii) a fragment, or derivative, or variant of said transcription or translation product, in a sample from said subject and comparing said level and/or said activity to a reference value representing a known disease or health status, thereby diagnosing or prognosticating said neurodegenerative disease in said subject, or determining whether said subject is at increased risk of developing said neurodegenerative disease.

2. A method of monitoring the progression of a neurodegenerative disease in a subject, comprising:
determining a level and/or an activity of
(i) a transcription product of a gene coding for human MAGUIN-1 and/or human MAGUIN-2, and/or
(ii) a translation product of a gene coding for human MAGUIN-1 and/or human MAGUIN-2, and/or
(iii) a fragment, or derivative, or variant of said transcription or translation product, in a sample from said subject and comparing said level and/or said activity to a reference value representing a known disease or health status, thereby monitoring the progression of said neurodegenerative disease in said subject.

3. A method of evaluating a treatment for a neurodegenerative disease, comprising:
determining a level and/or an activity of
(i) a transcription product of a gene coding for human MAGUIN-1 and/or human MAGUIN-2, and/or
(ii) a translation product of a gene coding for human MAGUIN-1 and/or human MAGUIN-2, and/or
(iii) a fragment, or derivative, or variant of said transcription or translation product, in a sample from a subject being treated for said disease and comparing said level and/or said activity to a reference value representing a known disease or health status, thereby evaluating said treatment for said neurodegenerative disease.

4. The method according to claim 1 wherein said neurodegenerative disease is Alzheimer's disease.

5. The method according to claim 1 wherein said sample is a cell, or a tissue, or a body fluid, in particular cerebrospinal fluid or blood.

6. The method according to claim 1 wherein said reference value is that of a level and/or an activity of
(i) a transcription product of a gene coding for human MAGUIN-1 and/or human MAGUIN-2, and/or
(ii) a translation product of a gene coding for human MAGUIN-1 and/or human MAGUIN-2, and/or
(iii) a fragment, or derivative, or variant of said transcription or translation product, in a sample from a subject not suffering from said neurodegenerative disease.

7. The method according to claim 1 wherein an alteration in the level and/or activity of a transcription product of the gene coding for human MAGUIN-1 and/or human MAGUIN-2 and/or a translation product of the gene coding for human MAGUIN-1 and/or human MAGUIN-2 and/or a fragment, or derivative, or variant thereof, in a sample cell, or tissue, or body fluid, in particular cerebrospinal fluid, from said subject relative to a reference value representing a known health status indicates a diagnosis, or prognosis, or increased risk of Alzheimer's disease in said subject.

8. The method according to claim 1, further comprising:
comparing a level and/or an activity of
(i) a transcription product of a gene coding for human MAGUIN-1 and/or human MAGUIN-2, and/or
(ii) a translation product of a gene coding for human MAGUIN-1 and/or human MAGUIN-2, and/or
(iii) a fragment, or derivative, or variant of said transcription or translation product, in a series of samples taken from said subject over a period of time.

9. The method according to claim 8, wherein said subject receives a treatment prior to one or more of said sample gatherings.

10. The method according to claim 9 wherein said level and/or activity is determined before and after said treatment of said subject.

11. A kit for diagnosing or prognosticating a neurodegenerative disease, in particular Alzheimer's disease, in a subject, or determining the propensity or predisposition of a subject to develop such a disease, said kit comprising:
(a) at least one reagent which is selected from the group consisting of
(i) reagents that selectively detect a transcription product of a gene coding for human MAGUIN-1 and/or human MAGUIN-2, and
(ii) reagents that selectively detect a translation product of a gene coding for human MAGUIN-1 and/or human MAGUIN-2 and
(b) an instruction for diagnosing or prognosticating a neurodegenerative disease, in particular Alzheimer's disease, or determining the propensity or predisposition of a subject to develop such a disease by
(i) detecting a level, or an activity, or both said level and said activity, of said transcription product and/or said translation product of a gene coding for human MAGUIN-1 and/or human MAGUIN-2 in a sample from said subject; and
(ii) diagnosing or prognosticating a neurodegenerative disease, in particular Alzheimer's disease, or determining the propensity or predisposition of said subject to develop such a disease,
wherein a varied level, or activity, or both said level and said activity, of said transcription product and/or said translation product compared to a reference value representing a known health status; or a level, or activity, or both said level and said activity, of said transcription product and/or said translation product similar or equal to a reference value representing a known disease status indicates a diagnosis or prognosis of a neurodegenerative disease, in particular Alzheimer's disease, or an increased propensity or predisposition of developing such a disease.

12. A method of treating or preventing a neurodegenerative disease, in particular Alzheimer's disease, in a subject comprising administering to said subject in a therapeutically or prophylactically effective amount an agent or agents which directly or indirectly affect an activity and/or a level of (i) a gene coding for human MAGUIN-1 and/or human MAGUIN-2, and/or (ii) a transcription product of a gene coding for human MAGUIN-1 and/or human MAGUIN-2, and/or (iii) a translation product of a gene coding for human MAGUIN-1 and/or human MAGUIN-2, and/or (iv) a fragment, or derivative, or variant of (i) to (iii).

13. A modulator of an activity and/or of a level of at least one substance which is selected from the group consisting of (i) a gene coding for human MAGUIN-1 and/or human MAGUIN-2, and/or (ii) a transcription product of a gene coding for human MAGUIN-1 and/or human MAGUIN-2, and/or (iii) a translation product of a gene coding for human MAGUIN-1 and/or human MAGUIN-2, and/or (iv) a fragment, or derivative, or variant of (i) to (iii).

14. A pharmaceutical composition comprising a modulator according to claim 13.

15. Use of a modulator of an activity and/or of a level of at least one substance which is selected from the group consisting of (i) a gene coding for human MAGUIN-1 and/or human MAGUIN-2, and/or (ii) a transcription product of a gene coding for human MAGUIN-1 and/or human MAGUIN-2, and/or (iii) a translation product of a gene coding for human MAGUIN-1 and/or human MAGUIN-2, and/or (iv) a fragment, or derivative, or variant of (i) to (iii) for a preparation of a medicament for treating or preventing a neurodegenerative disease, in particular Alzheimer's disease.

16. A recombinant, non-human animal comprising a non-native gene sequence coding for human MAGUIN-1 and/or human MAGUIN-2 or a fragment, or a derivative, or a variant thereof, said animal being obtainable by:
(i) providing a gene targeting construct comprising said gene sequence and a selectable marker sequence, and
(ii) introducing said targeting construct into a stem cell of a non-human animal, and
(iii) introducing said non-human animal stem cell into a non-human embryo, and
(iv) transplanting said embryo into a pseudopregnant non-human animal, and
(v) allowing said embryo to develop to term, and
(vi) identifying a genetically altered non-human animal whose genome comprises a modification of said gene sequence in both alleles, and
(vii) breeding the genetically altered non-human animal of step (vi) to obtain a genetically altered non-human animal whose genome comprises a modification of said endogenous gene, wherein said disruption results in said non-human animal exhibiting a predisposition to developing symptoms of a neurodegenerative disease or related diseases or disorders.

17. Use of the recombinant, non-human animal according to claim 16 for screening, testing, and validating compounds, agents, and modulators in the development of diagnostics and therapeutics to treat neurodegenerative diseases, in particular Alzheimer's disease.

18. An assay for screening for a modulator of neurodegenerative diseases, in particular Alzheimer's disease, or related diseases or disorders of one or more substances selected from the group consisting of
(i) a gene coding for human MAGUIN-1 and/or human MAGUIN-2, and/or
(ii) a transcription product of a gene coding for human MAGUIN-1 and/or human MAGUIN-2, and/or
(iii) a translation product of a gene coding for human MAGUIN-1 and/or human MAGUIN-2, and/or
(iv) a fragment, or derivative, or variant of (i) to (iii), said method comprising:
(a) contacting a cell with a test compound;
(b) measuring the activity and/or level of one or more substances recited in (i) to (iv);
(c) measuring the activity and/or level of one or more substances recited in (i) to (iv) in a control cell not contacted with said test compound; and
(d) comparing the levels and/or activities of the substance in the cells of step (b) and (c), wherein an alteration in the activity and/or level of substances in the contacted cells indicates that the test compound is a modulator of said diseases or disorders.

19. A method of testing a compound, preferably of screening a plurality of compounds, for inhibition of binding between a ligand and human MAGUIN-1 and/or human MAGUIN-2, or fragments, or derivatives, or variants thereof, said method comprising the steps of:
(i) adding a liquid suspension of said human MAGUIN-1 and/or human MAGUIN-2, or fragments, or derivatives, or variants thereof, to a plurality of containers;
(ii) adding a compound, preferably a plurality of compounds, to be screened for said inhibition of binding to said plurality of containers;
(iii) adding a detectable ligand, in particular a fluorescently detectable ligand, to said containers;
(iv) incubating the liquid suspension of said human MAGUIN-1 and/or human MAGUIN-2, or said fragments, or derivatives, or variants thereof, and said compound, preferably said plurality of compounds, and said ligand;
(v) measuring amounts of detectable ligand or fluorescence associated with said human MAGUIN-1 and/or human MAGUIN-2, or with said fragments, or derivatives, or variants thereof; and
(vi) determining the degree of inhibition by one or more of said compounds of binding of said ligand to said human MAGUIN-1 and/or human MAGUIN-2, or said fragments, or derivatives, or variants thereof.

20. A method of testing a compound, preferably of screening a plurality of compounds, to determine the degree of binding of said compound or compounds to human MAGUIN-1 and/or human MAGUIN-2, or to fragments, or derivatives, or variants thereof, said method comprising the steps of:
(i) adding a liquid suspension of said human MAGUIN-1 and/or human MAGUIN-2, or fragments, or derivatives, or variants thereof, to a plurality of containers;
(ii) adding a detectable compound, preferably a plurality of detectable compounds, in particular fluorescently detectable compounds, to be screened for said binding to said plurality of containers;
(iii) incubating the liquid suspension of said human MAGUIN-1 and/or human MAGUIN-2, or said fragments, or derivatives, or variants thereof, and said compound, preferably said plurality of compounds;
(iv) measuring amounts of detectable compound or fluorescence associated with said human MAGUIN-1 and/or human MAGUIN-2, or with said fragments, or derivatives, or variants thereof; and
(v) determining the degree of binding by one or more of said compounds to said human MAGUIN-1 and/or human MAGUIN-2, or said fragments, or derivatives, or variants thereof.

21. A method for producing a medicament comprising the steps of (i) identifying a modulator of neurodegenerative diseases, in particular Alzheimer's disease, by a method according to claim 18 and (ii) admixing the modulator with a pharmaceutical carrier.

22. A method for producing a medicament comprising the steps of (i) identifying a compound as an inhibitor of binding between a ligand and a human MAGUIN-1 and/or human MAGUIN-2 gene product by a method according to claim 19 and (ii) admixing the compound with a pharmaceutical carrier.

23. A method for producing a medicament comprising the steps of (i) identifying a compound as a binder to a human MAGUIN-1 and/or human MAGUIN-2 gene product by a method according to claim 20 and (ii) admixing the compound with a pharmaceutical carrier.

24. A medicament obtainable by any of the methods according to claim 21.

25. A medicament obtained by any of the methods according to claim 21.

26. A protein molecule shown in SEQ ID NO.1 or SEQ ID NO.2, or a fragment, or derivative, or variant thereof, for use as a diagnostic target for detecting a neurodegenerative disease, preferably Alzheimer's disease.

27. A protein molecule shown in SEQ ID NO. 1 or SEQ ID NO.2, or a fragment, or derivative, or variant thereof, for use as a screening target for reagents or compounds preventing, or treating, or ameliorating a neurodegenerative disease, preferably Alzheimer's disease.

28. An antibody specifically immunoreactive with an immunogen, wherein said immunogen is a protein molecule shown in SEQ ID NO. 1, or a fragment, or derivative, or variant thereof.

29. An antibody specifically immunoreactive with an immunogen, wherein said immunogen is a protein molecule shown in SEQ ID NO. 2, or a fragment, or derivative, or variant thereof.

30. Use of an antibody of claim 28, for detecting the pathological state of a cell in a sample from a subject, comprising immunocytochemical staining of said cell with said antibody, wherein an altered degree of staining, or an altered staining pattern in said cell compared to a cell representing a known health status indicates a pathological state of said cell.


SEQUENCE LISTING (24 sequences)

SEQ ID NO: 1 - protein, 1034 aa, Homo sapiens
MALIMEPVSKWSPSQVVDWMKGLDDCLQQYIKNFEREKISGDQLLRITHQELEDLGVSRIGHQELILEAVDLLCALNYGLETENLKTLSHKLNASAKNLQNFITGRRRSGHYDGRTSRKLPNDFLTSVVDLIGAAKSLLAWLDRSPFAAVTDYSVTRNNVIQLCLELTTIVQQDCTVYETENKILHVCKTLSGVCDHIISLSSDPLVSQSAHLEVIQLANIKPSEGLGMYIKSTYDGLHVITGTTENSPADRCKKIHAGDEVIQVNHQTVVGWQLKNLVNALREDPSGVILTLKKRPQSMLTSAPALLKNMRWKPLALQPLIPRSPTSSVATPSSTISTPTKRDSSALQDLYIPPPPAEPYIPRDEKGNLPCEDLRGHMVGKPVHKGSESPNSFLDQEYRKRFNIVEEDTVLYCYEYEKGRSSSQGRRESTPTYGKLRPISMPVEYNWVGDYEDPNKMKRDSRRENSLLRYMSNEKIAQEEYMFQRNSKKDTGKKSKKKGDKSNSPTHYSLLPSLQMDALRQDIMGTPVPETTLYHTFQQSSLQHKSKKKNKGPIAGKSKRRISCKDLGRGDCEGWLWKKKDAKSYFSQKWKKYWFVLKDASLYWYINEEDEKAEGFISLPEFKIDRASECRKKYAFKACHPKIKSFYFAAEHLDDMNRWLNRINMLTAGYAERERIKQEQDYWSESDKEEADTPSTPKQDSPPPPYDTYPRPPSMSCASPYVEAKHSRLSSTETSQSQSSHEEFRQEVTGSSAVSPIRKTASQRRSWQDLIETPLTSSGLHYLQTLPLEDSVFSDSAAISPEHRRQSTLPTQKCHLQDHYGPYPLAESERMQVLNGNGGKPRSFTLPRDSGFNHCCLNAPVSACDPQDDVQPPEVEEEEEEEEEEGEAAGENIGEKSESREEKLGDSLQDLYRALEQASLSPLGEHRISTKMEYKLSFIKRCNDPVMNEKLHRLRILKSTLKAREGEVAIIDKVLDNPDLTSKEFQQWKQMYLDLFLDICQNTTSNDPLSISSEVDVITSSLAHTHSYIETHV

SEQ ID NO: 2 - protein, 948 aa, Homo sapiens
FIGRESEQIDNAMINACIDSEQENCEFHMANMAGINPRTEINLENGTHAAMALIMEPVSKWSPSQVVDWMKGLDDCLQQYIKNFEREKISGDQLLRITHQELEDLGVSRIGHQELILEAVDLLCALNYGLETENLKTLSHKLNASAKNLQNFITGRRRSGHYDGRTSRKLPNDFLTSVVDLIGAAKSLLAWLDRSPFAAVTDYSVTRNNVIQLCLELTTIVQQDCTVYETENKILHVCKTLSGVCDHIISLSSDPLVSQSAHLEVIQLANIKPSEGLGMYIKSTYDGLHVITGTTENSPADRCKKIHAGDEVIQVNHQTVVGWQLKNLVNALREDPSGVILTLKKRPQSMLTSAPALLKNMRWKPLALQPLIPRSPTSSVATPSSTISTPTKRDSSALQDLYIPPPPAEPYIPRDEKGNLPCEDLRGHMVGKPVHKGSESPNSFLDQEYRKRFNIVEEDTVLYCYEYEKGRSSSQGRRESTPTYGKLRPISMPVEYNWVGDYEDPNKMKRDSRRENSLLRYMSNEKIAQEEYMFQRNSKKDTGKKSKKKGDKSNSPTHYSLLPSLQMDALRQDIMGTPVPETTLYHTFQQSSLQHKSKKKNKGPIAGKSKRRISCKDLGRGDCEGWLWKKKDAKSYFSQKWKKYWFVLKDASLYWYINEEDEKAEGFISLPEFKIDRASECRKKYAFKACHPKIKSFYFAAEHLDDMNRWLNRINMLTAGYAERERIKQEQDYWSESDKEEADTPSTPKQDSPPPPYDTYPRPPSMSCASPYVEAKHSRLSSTETSQSQSSHEEFRQEVTGSSAVSPIRKTASQRRSWQDLIETPLTSSGLHYLQTLPLEDSVFSDSAAISPEHRRQSTLPTQKCHLQDHYGPYPLAESERMQVLNGNGGKPRSFTLPRDSGFNHCCLNAPVSACDPQDDVQPPEVEEEEEEEEEEGEAAGENIGEKS

SEQ ID NO: 3 - DNA, 3105 bp, Homo sapiens
atggctctgataatggaaccggtgagcaaatggtctccgagtcaagtagtggactggatgaaaggtcttgatgactgtttgcagcagtatattaagaactttgagagggagaagatcagtggggaccagctgctgcgcattacacatcaggagctagaagatctgggggtcagccgcattggccatcaggaactgatcttggaagcagttgaccttctgtgtgcattgaattatggcttggaaacagaaaatctaaaaaccctttctcacaagttgaatgcatctgccaaaaatctgcagaattttataacaggaaggagaaggagtggccattatgatgggaggaccagccgaaaattgccaaacgactttctgacctcagttgtggatctgattggagcagccaagagtctgcttgcctggttggacaggtcaccatttgctgctgtgacagactattcagttacaagaaataatgtcatacaactctgcctggagttaacaacaattgtgcaacaggattgtactgtatatgaaacagagaataaaattcttcacgtgtgtaaaactctttctggagtctgtgaccacatcatatccctgtcgtcagatcctctggtttcacagtctgctcacctggaagtgattcaactggcaaacattaaaccaagcgaagggctgggtatgtatattaaatctacatatgatggcctccatgtaattactggaaccacagaaaattcacctgcagatcggtgcaagaaaatccatgctggcgatgaagtgattcaagttaatcatcagactgtggtggggtggcagttgaaaaatttggtgaatgcactacgagaggacccgagtggtgttatcttaactttgaaaaagcgacctcagagcatgcttacctcagcaccagctttactgaaaaatatgagatggaagccccttgctctgcagcctcttatacctagaagtcccacaagcagcgttgccacgccttccagcaccatcagtacacccaccaaaagagacagttctgccctccaggatctctacattccccctcctcctgcagaaccatatattcccagggatgaaaaaggaaaccttccttgtgaagacctcagaggacatatggtgggcaagccagtgcataagggatctgaatcaccaaattcatttctggatcaggaatatcgaaagagatttaatattgtcgaagaagatactgtcttatattgctatgaatatgaaaaaggaagatcaagtagtcaaggaagacgagaaagcaccccaacttatggcaagctacgacctatatctatgccagtggaatataattgggtgggggactatgaagatccaaataagatgaagagagatagtagaagagaaaactctctacttcggtatatgagcaatgaaaagattgctcaagaagaatacatgtttcagagaaacagcaaaaaggacacagggaagaagtcaaaaaagaagggtgataagagtaatagcccaactcactattcattgctacctagtttacaaatggatgcactgagacaagacatcatgggcactcctgtgccagagaccacactataccatacatttcagcagtcctcactgcagcacaaatcaaagaagaaaaacaaaggtcctatagcaggcaagagcaaaagacgaatttcttgcaaagatcttggccgtggtgactgtgagggctggctttggaaaaagaaagatgcgaagagttacttttcacagaaatggaaaaaatattggtttgtcctaaaggatgcatccctttattggtatattaatgaggaggatgaaaaagcagaaggattcattagcctgcctgaatttaaaattgatagagccagtgaatgccgcaaaaaatatgcattcaaagcctgtcatcctaaaatcaaaagcttttattttgctgctgaacatcttgatgatatgaacaggtggcttaacagaattaatatgctgactgcaggatatgcagaaagagagaggattaagcaggaacaagattactggagtgagagtgacaaggaagaagcagatactccatcaacaccaaaacaagatagccctccacccccatatgatacatacccacgacctccctcgatgagttgcgccagtccttatgtggaagcaaaacatagccgactttcctccacggagacttctcagtctcagtcttctcatgaggagtttcgccaggaagtaactgggagcagtgcagtgtctcccattcgcaagacagccagtcagcgccgctcctggcaggatttaattgagacgccactgacaagttcaggcttacactatcttcagactctgcccctggaggattctgtcttctctgactccgcggccatctccccagagcacaggcggcagtctaccctgccaactcagaaatgccacctgcaggatcactatgggccataccccttagctgagagtgagaggatgcaagtgctaaatggaaatgggggcaagcctcgaagttttactctgcctcgagatagcgggttcaaccattgctgtctgaatgctccagttagtgcctgtgacccacaggatgacgtgcaacccccagaggtggaggaagaggaggaggaggaggaggaggaaggggaggcagcaggggaaaacataggagaaaaaagtgaaagcagagaagaaaagttaggagactcattgcaagatttatacagggcactggagcaggccagtctgtcaccactaggagaacatcgtatttcaaccaagatggaatacaagctatcatttataaaaagatgtaatgatcctgtaatgaatgaaaaactacaccggctgagaattctcaaaagcactttaaaggccagagaaggggaagtagccattatcgataaagtcctagacaatccagacttgacatctaaagaattccaacaatggaagcagatgtacctcgaccttttcttggatatctgtcaaaataccacctcaaatgacccactgagtatttcttctgaagtagatgtaatcacttcctctctagcacacactcattcatacattgaaacgcatgtctaa

SEQ ID NO: 4 - DNA, 2683 bp, Homo sapiens
atggctctgataatggaaccggtgagcaaatggtctccgagtcaagtagtggactggatgaaaggtcttgatgactgtttgcagcagtatattaagaactttgagagggagaagatcagtggggaccagctgctgcgcattacacatcaggagctagaagatctgggggtcagccgcattggccatcaggaactgatcttggaagcagttgaccttctgtgtgcattgaattatggcttggaaacagaaaatctaaaaaccctttctcacaagttgaatgcatctgccaaaaatctgcagaattttataacaggaaggagaaggagtggccattatgatgggaggaccagccgaaaattgccaaacgactttctgacctcagttgtggatctgattggagcagccaagagtctgcttgcctggttggacaggtcaccatttgctgctgtgacagactattcagttacaagaaataatgtcatacaactctgcctggagttaacaacaattgtgcaacaggattgtactgtatatgaaacagagaataaaattcttcacgtgtgtaaaactctttctggagtctgtgaccacatcatatccctgtcgtcagatcctctggtttcacagtctgctcacctggaagtgattcagctggcaaacattaaaccaagcgaagggctgggtatgtatattaaatctacatatgatggcctccatgtaattactggaaccacagaaaattcacctgcagatcggtgcaagaaaatccatgctggcgatgaagtgattcaagttaatcatcagactgtggtggggtggcagttgaaaaatttggtgaatgcactacgagaggacccgagtggtgttatcttaactttgaaaaagcgacctcagagcatgcttacctcagcaccagctttactgaaaaatatgagatggaagccccttgctctgcagcctcttatacctagaagtcccacaagcagcgttgccacgccttccagcaccatcagtacacccaccaaaagagacagttctgccctccaggatctctacattccccctcctcctgcagaaccatatattcccagggatgaaaaaggaaaccttccttgtgaagacctcagaggacatatggtgggcaagccagtgcataagggatctgaatcaccaaattcatttctggatcaggaatatcgaaagagatttaatattgtcgaagaagatactgtcttatattgctatgaatatgaaaaaggaagatcaagtagtcaaggaagacgagaaagcaccccaacttatggcaagctacgacctatatctatgccagtggaatataattgggtgggggactatgaagatccaaataagatgaagagagatagtagaagagaaaactctctacttcggtatatgagcaatgaaaagattgctcaagaagaatacatgtttcagagaaacagcaaaaaggacacagggaagaagtcaaaaaagaagggtgataagagtaatagcccaactcactattcattgctacctagtttacaaatggatgcactgagacaagacatcatgggcactcctgtgccagagaccacactataccatacatttcagcagtcctcactgcagcacaaatcaaagaagaaaaacaaaggtcctatagcaggcaagagcaaaagacgaatttcttgcaaagatcttggccgtggtgactgtgagggctggctttggaaaaagaaagatgcgaagagttacttttcacagaaatggaaaaaatattggtttgtcctaaaggatgcatccctttattggtatattaatgaggaggatgaaaaagcagaaggattcattagcctgcctgaatttaaaattgatagagccagtgaatgccgcaaaaaatatgcattcaaagcctgtcatcctaaaatcaaaagcttttattttgctgctgaacatcttgatgatatgaacaggtggcttaacagaattaatatgctgactgcaggatatgcagaaagagagaggattaagcaggaacaagattactggagtgagagtgacaaggaagaagcagatactccatcaacaccaaaacaagatagccctccacccccatatgatacatacccacgacctccctcgatgagttgcgccagtccttatgtggaagcaaaacatagccgactttcctccacggagacttctcagtctcagtcttctcatgaggagtttcgccaggaagtaactgggagcagtgcagtgtctcccattcgcaagacagccagtcagcgccgctcctggcaggatttaattgagacgccactgacaagttcaggcttacactatcttcagactctgcccctggaggattctgtcttctctgactccgcggccatctccccagagcacaggcggcagtctaccctgccaactcagaaatgccacctgcaggatcactatgggccataccccttagctgagagtgagaggatgcaagtgctaaatggaaatgggggcaagcctcgaagttttactctgcctcgagatagcgggttcaaccattgctgtctgaatgctccagttagtgcctgtgacccacaggatgacgtgcaacccccagaggtggaggaagaggaggaggaggaggaggaggaaggggaggcagcaggggaaaacatag

SEQ ID NO: 5 - DNA, 5749 bp, Artificial Sequence
  Description of Artificial Sequence cDNA of human Maguin-1
cgggcagctagtcgtgctcggggcttcactcccgcgcgtgaggcgagcgggcaagttggctgagggcgtgcggcagaggctgcttccctcggcgacgcgacccctcagcaactcaagctatgaactgaagctccctagggacggagaccggagcggagcggcggaggcagcagcagcagcagcagcagcagcagcagcagcagccgccgccgccgccgccttagcgggaactgagcagacccggcgcggagccacgactcctgcacgtttacctccctgtcgccgttcctgccggcggttggctaaaagacgttacagccgcgagacccgacacacaaaagccgctttctccgcgccgcccgcccagggaggctgcggccagcaagggaccccacctgagagcagctcgggctgctgagttcgttttgtgtctgagctctgcgctctgcacggaaccgaccccgtacccatggctctgataatggaaccggtgagcaaatggtctccgagtcaagtagtggactggatgaaaggtcttgatgactgtttgcagcagtatattaagaactttgagagggagaagatcagtggggaccagctgctgcgcattacacatcaggagctagaagatctgggggtcagccgcattggccatcaggaactgatcttggaagcagttgaccttctgtgtgcattgaattatggcttggaaacagaaaatctaaaaaccctttctcacaagttgaatgcatctgccaaaaatctgcagaattttataacaggaaggagaaggagtggccattatgatgggaggaccagccgaaaattgccaaacgactttctgacctcagttgtggatctgattggagcagccaagagtctgcttgcctggttggacaggtcaccatttgctgctgtgacagactattcagttacaagaaataatgtcatacaactctgcctggagttaacaacaattgtgcaacaggattgtactgtatatgaaacagagaataaaattcttcacgtgtgtaaaactctttctggagtctgtgaccacatcatatccctgtcgtcagatcctctggtttcacagtctgctcacctggaagtgattcaactggcaaacattaaaccaagcgaagggctgggtatgtatattaaatctacatatgatggcctccatgtaattactggaaccacagaaaattcacctgcagatcggtgcaagaaaatccatgctggcgatgaagtgattcaagttaatcatcagactgtggtggggtggcagttgaaaaatttggtgaatgcactacgagaggacccgagtggtgttatcttaactttgaaaaagcgacctcagagcatgcttacctcagcaccagctttactgaaaaatatgagatggaagccccttgctctgcagcctcttatacctagaagtcccacaagcagcgttgccacgccttccagcaccatcagtacacccaccaaaagagacagttctgccctccaggatctctacattccccctcctcctgcagaaccatatattcccagggatgaaaaaggaaaccttccttgtgaagacctcagaggacatatggtgggcaagccagtgcataagggatctgaatcaccaaattcatttctggatcaggaatatcgaaagagatttaatattgtcgaagaagatactgtcttatattgctatgaatatgaaaaaggaagatcaagtagtcaaggaagacgagaaagcaccccaacttatggcaagctacgacctatatctatgccagtggaatataattgggtgggggactatgaagatccaaataagatgaagagagatagtagaagagaaaactctctacttcggtatatgagcaatgaaaagattgctcaagaagaatacatgtttcagagaaacagcaaaaaggacacagggaagaagtcaaaaaagaagggtgataagagtaatagcccaactcactattcattgctacctagtttacaaatggatgcactgagacaagacatcatgggcactcctgtgccagagaccacactataccatacatttcagcagtcctcactgcagcacaaatcaaagaagaaaaacaaaggtcctatagcaggcaagagcaaaagacgaatttcttgcaaagatcttggccgtggtgactgtgagggctggctttggaaaaagaaagatgcgaagagttacttttcacagaaatggaaaaaatattggtttgtcctaaaggatgcatccctttattggtatattaatgaggaggatgaaaaagcagaaggattcattagcctgcctgaatttaaaattgatagagccagtgaatgccgcaaaaaatatgcattcaaagcctgtcatcctaaaatcaaaagcttttattttgctgctgaacatcttgatgatatgaacaggtggcttaacagaattaatatgctgactgcaggatatgcagaaagagagaggattaagcaggaacaagattactggagtgagagtgacaaggaagaagcagatactccatcaacaccaaaacaagatagccctccacccccatatgatacatacccacgacctccctcgatgagttgcgccagtccttatgtggaagcaaaacatagccgactttcctccacggagacttctcagtctcagtcttctcatgaggagtttcgccaggaagtaactgggagcagtgcagtgtctcccattcgcaagacagccagtcagcgccgctcctggcaggatttaattgagacgccactgacaagttcaggcttacactatcttcagactctgcccctggaggattctgtcttctctgactccgcggccatctccccagagcacaggcggcagtctaccctgccaactcagaaatgccacctgcaggatcactatgggccataccccttagctgagagtgagaggatgcaagtgctaaatggaaatgggggcaagcctcgaagttttactctgcctcgagatagcgggttcaaccattgctgtctgaatgctccagttagtgcctgtgacccacaggatgacgtgcaacccccagaggtggaggaagaggaggaggaggaggaggaggaaggggaggcagcaggggaaaacataggagaaaaaagtgaaagcagagaagaaaagttaggagactcattgcaagatttatacagggcactggagcaggccagtctgtcaccactaggagaacatcgtatttcaaccaagatggaatacaagctatcatttataaaaagatgtaatgatcctgtaatgaatgaaaaactacaccggctgagaattctcaaaagcactttaaaggccagagaaggggaagtagccattatcgataaagtcctagacaatccagacttgacatctaaagaattccaacaatggaagcagatgtacctcgaccttttcttggatatctgtcaaaataccacctcaaatgacccactgagtatttcttctgaagtagatgtaatcacttcctctctagcacacactcattcatacattgaaacgcatgtctaaatgtattctgccttcagaccatctagtacctgctggtactctgaacaagtatataaggtagtttttatatcaatgtgtggaacacttgacaagctatactttaatgttaccaaactatatgaaacaaaccatatatggtcacaataccactatctttaatgagcatttgtatattttatatgcaacagtgctcagcttatgtttaccatgtgcaaaatcaactgtctttaatgacttaaaattaacttttgcaaacaattctaaatacaggtggtcttcaagtagtaaaaccacaaaaggcagttttctatctatggtcatcttttctccctttaagttaattttatataaacaagacttcaaaagtaaatcacattttttcaggtgcagacatccttgtgggtgggaaagaatttaaaccttttttatatttattaaaatgttctaagaattttcttaaacattgcacaaagtttaatgctgtagttttatttttgtgaaatgtagatgcgcatacaagagctaagcaaaatagaagagcatcgacataagaaaagttcaggtatctaatattcgtcttaatagtctattaacttgtgaaagctaagttaatggaaatattattccaaatctatgagaacacttggtgtatcagggcaaagctttgtaagatgtttttgtaactaagaccaaaattgaagatagagctgctttattttcttggtttaaatcttcctttatttttgtagtgatgagatgctgattgtgtacagaagaatttgagaggggatttttaaaaactgacttaacacacccagaaaggcagctaacagctatatatatatataaatttcagcccaaactcatgtttttaaactccaactcttaaaagacaacaaggtataaactgaaatgaatcaactttccacttagtttccaattttcccctagtccactaattaaacttaggtaattatacttcaggtagggaagtacaatatgtttagtttcaggctgatgtgtgttataaaaaacaacactgaaaaataaaaatgtacttcccttctaaggagcaagcaggtgatggtcattcaaagagatgtcacattgaattatgagagaaacaatttagaggtttttttcctggcttcatgaattgttctatagagtggatgaagtctaaggaaaagtcctcttcatatatttccatttataagcgtcttgtttttgaaagtgatcacagcatgaaaatgactgtgctgctttttagtgtctggctgcataatgtacaagtcacaatttgctgtttttttcaggaggagaaagggaacctcctttactattctatatcctaaaatctacttctaatcagctttatactgttgcctgtacagctcagtgaatgtactttcatctttaagagttcagatatatgccagtgaatatttttgctgtagaggagaaagtaaaaactccacagcggggatctttttctttgcttttgaaaccaccattgaatcactatcgttttgcagactttgcacaactgtacaggagagtggcctttctacagcacattttcagtaatcctatatttagtcaaaatggatgagaaatcatgtattaatgtttgtatggaattttgggtccagtgtaatatttttatcatttaaaaagaactctatttgtaaaaacatttatttactgcatggatattgacgcacattaaatttgtgggattttgtatatgtaaaaaaaaaaaaaaaaaaaaaaaacaaaaaacctcttgtcctaaaatgaagtgtgcttgttaacaggtgtttagacttattgatgtttactagaccaaatgtgtatgttcacttaaaaatatatgtacctgatggatgtgtcatgtttacagtggccaggttgtggcctgtaaacagcaagcagttgacgggaagactagctctgttgctactaagcagcttttacttttgtaaagtcagctctgttgttttaaatggtaaaaattaaactaatgaatttgacaagactcgtggctagcctagcatgaaagagaccttttaacactatataatatctgtacattttattgcattcgtttcaaatctaggagagaggcagcactgtaaactgaagtcaaataaattcagctcttaatgaatcctt

SEQ ID NO: 6 - DNA, 4350 bp, Artificial Sequence
  Description of Artificial Sequence cDNA of human Maguin-2
gtgctcggggcttcactcccgcgcgtgaggcgagcgggcaagttggctgagggcgtgcggcagaggctgcttccctcggcgacgcgacccctcagcaactcaagctatgaactgaagctccctagggacggagaccggagcggagcggcggaggcagcagcagcagcagcagcagcagcagcagcagcagccgccgccgccgccgccttagcgggaactgagcagacccggcgcggagccacgactcctgcacgtttacctccctgtcgccgttcctgccggcggttggctaaaagacgttacagccgcgagacccgacacacaaaagccgctttctccgcgccgcccgcccagggaggctgcggccagcaagggaccccacctgagagcagctcgggctgctgagttcgttttgtgtctgagctctgcgctctgcacggaaccgaccccgtacccatggctctgataatggaaccggtgagcaaatggtctccgagtcaagtagtggactggatgaaaggtcttgatgactgtttgcagcagtatattaagaactttgagagggagaagatcagtggggaccagctgctgcgcattacacatcaggagctagaagatctgggggtcagccgcattggccatcaggaactgatcttggaagcagttgaccttctgtgtgcattgaattatggcttggaaacagaaaatctaaaaaccctttctcacaagttgaatgcatctgccaaaaatctgcagaattttataacaggaaggagaaggagtggccattatgatgggaggaccagccgaaaattgccaaacgactttctgacctcagttgtggatctgattggagcagccaagagtctgcttgcctggttggacaggtcaccatttgctgctgtgacagactattcagttacaagaaataatgtcatacaactctgcctggagttaacaacaattgtgcaacaggattgtactgtatatgaaacagagaataaaattcttcacgtgtgtaaaactctttctggagtctgtgaccacatcatatccctgtcgtcagatcctctggtttcacagtctgctcacctggaagtgattcagctggcaaacattaaaccaagcgaagggctgggtatgtatattaaatctacatatgatggcctccatgtaattactggaaccacagaaaattcacctgcagatcggtgcaagaaaatccatgctggcgatgaagtgattcaagttaatcatcagactgtggtggggtggcagttgaaaaatttggtgaatgcactacgagaggacccgagtggtgttatcttaactttgaaaaagcgacctcagagcatgcttacctcagcaccagctttactgaaaaatatgagatggaagccccttgctctgcagcctcttatacctagaagtcccacaagcagcgttgccacgccttccagcaccatcagtacacccaccaaaagagacagttctgccctccaggatctctacattccccctcctcctgcagaaccatatattcccagggatgaaaaaggaaaccttccttgtgaagacctcagaggacatatggtgggcaagccagtgcataagggatctgaatcaccaaattcatttctggatcaggaatatcgaaagagatttaatattgtcgaagaagatactgtcttatattgctatgaatatgaaaaaggaagatcaagtagtcaaggaagacgagaaagcaccccaacttatggcaagctacgacctatatctatgccagtggaatataattgggtgggggactatgaagatccaaataagatgaagagagatagtagaagagaaaactctctacttcggtatatgagcaatgaaaagattgctcaagaagaatacatgtttcagagaaacagcaaaaaggacacagggaagaagtcaaaaaagaagggtgataagagtaatagcccaactcactattcattgctacctagtttacaaatggatgcactgagacaagacatcatgggcactcctgtgccagagaccacactataccatacatttcagcagtcctcactgcagcacaaatcaaagaagaaaaacaaaggtcctatagcaggcaagagcaaaagacgaatttcttgcaaagatcttggccgtggtgactgtgagggctggctttggaaaaagaaagatgcgaagagttacttttcacagaaatggaaaaaatattggtttgtcctaaaggatgcatccctttattggtatattaatgaggaggatgaaaaagcagaaggattcattagcctgcctgaatttaaaattgatagagccagtgaatgccgcaaaaaatatgcattcaaagcctgtcatcctaaaatcaaaagcttttattttgctgctgaacatcttgatgatatgaacaggtggcttaacagaattaatatgctgactgcaggatatgcagaaagagagaggattaagcaggaacaagattactggagtgagagtgacaaggaagaagcagatactccatcaacaccaaaacaagatagccctccacccccatatgatacatacccacgacctccctcgatgagttgcgccagtccttatgtggaagcaaaacatagccgactttcctccacggagacttctcagtctcagtcttctcatgaggagtttcgccaggaagtaactgggagcagtgcagtgtctcccattcgcaagacagccagtcagcgccgctcctggcaggatttaattgagacgccactgacaagttcaggcttacactatcttcagactctgcccctggaggattctgtcttctctgactccgcggccatctccccagagcacaggcggcagtctaccctgccaactcagaaatgccacctgcaggatcactatgggccataccccttagctgagagtgagaggatgcaagtgctaaatggaaatgggggcaagcctcgaagttttactctgcctcgagatagcgggttcaaccattgctgtctgaatgctccagttagtgcctgtgacccacaggatgacgtgcaacccccagaggtggaggaagaggaggaggaggaggaggaggaaggggaggcagcaggggaaaacataggagaaaaaagctaatacactgcgagagttggtagaacctctccatgccaaatcggatccacttctgttggcactcaacccattggactcacagattgataagctaatgtttagagaatttagatcggagagagtcggtacggcgcagactcaacatcaacctcttgcaagcaactaaaatggcctcgtccttgctgtttataacagaaaacagacttgtaaaaagcttagatcatcaagtgttttggattgggggcctcccaaagggatataagaggggcaggccactcttaagaagaatgcgagctttctacattgggactagcataagatcaaagccaatcaagatggagcacagtaacagaaaactgcggtttctgtgggagaacagaaggggaaagggtcttaactgggaaagggctctgtgtggtaacacctcagttgtgttctcctgacaccaggaaaagagagggatcagcttcaataactagaaaattctggctgtttaatggactctttggtggcctctttaaggcaaagcagagaaagcaaattatgtattaagtgtattttgcatttttaaaacttgacgtgctgtattgtactaaattaagtgtaatctattaaggcaaggtatacacaatttgctttgaaacttactatgtttattctattataaagtgtattcaggtgcaacacagagactgctttcggtgacattaatgaagaaaatttctcatgccaggctttattatagaatcttcagctaaaatcctaactttctccttatttcttggcacttgtatacaagtggtgttgcctcttagggcaggcatgagctattcttttctgtaaaatattttgaatctataggctgtgggtttcatttttgaaaagtattttgtctggatgtctttcaaactagcttcagatattatttaatactatgtaactgggtcccctatggctcaatcaatattgcttatttttcttctgtagtggatgtgaaatttcctttagttggataagatacactgtaataattttaatgctaattaatgatatttcatactgtgcaatgaacagataatttaacactgtattttgaaatgtttttttcttcctgtcaccgcagtgtgtggtattgcataatgtgaatacctgtaaaaatataaattacttaaaaataaaaatatgaccaattggtatcag

SEQ ID NO: 7 - DNA, 50 bp, Artificial Sequence
  Description of Artificial Sequence cDNA fragment of Maguin-1
ggagagaggcagcactgtaaactgaagtcaaataaattcagctcttaatg

SEQ ID NO: 8 - DNA, 13 bp, Artificial Sequence
  Description of Artificial Sequence 0ne-base-anchor oligonucleotides
httttttttttta

SEQ ID NO: 9 - DNA, 13 bp, Artificial Sequence
  Description of Artificial Sequence 0ne-base-anchor oligonucleotides
htttttttttttg

SEQ ID NO: 10 - DNA, 13 bp, Artificial Sequence
  Description of Artificial Sequence 0ne-base-anchor oligonucleotides
htttttttttttc

SEQ ID NO: 11 - DNA, 19 bp, Artificial Sequence
  Description of Artificial Sequence Primer for Maguin-1
cagcaagcagttgacggga

SEQ ID NO: 12 - DNA, 22 bp, Artificial Sequence
  Description of Artificial Sequence Primer for Maguin-1
tgaatttgacaagactcgtggc

SEQ ID NO: 13 - DNA, 19 bp, Artificial Sequence
  Description of Artificial Sequence Primer for Maguin-2
gggcctcccaaagggatat

SEQ ID NO: 14 - DNA, 22 bp, Artificial Sequence
  Description of Artificial Sequence Primer for Maguin-2
cccaatgtagaaagctcgcatt

SEQ ID NO: 15 - DNA, 20 bp, Artificial Sequence
  Description of Artificial Sequence primer for cyclophilin B
actgaagcactacgggcctg

SEQ ID NO: 16 - DNA, 19 bp, Artificial Sequence
  Description of Artificial Sequence primer for cyclophilin B
agccgttggtgtctttgcc

SEQ ID NO: 17 - DNA, 20 bp, Artificial Sequence
  Description of Artificial Sequence Primer for ribosomal protein S9
ggtcaaatttaccctggcca

SEQ ID NO: 18 - DNA, 22 bp, Artificial Sequence
  Description of Artificial Sequence Primer for ribosomal protein S9
tctcatcaagcgtcagcagttc

SEQ ID NO: 19 - DNA, 19 bp, Artificial Sequence
  Description of Artificial Sequence primer for beta-actin
tggaacggtgaaggtgaca

SEQ ID NO: 20 - DNA, 19 bp, Artificial Sequence
  Description of Artificial Sequence primer for beta-actin
ggcaagggacttcctgtaa

SEQ ID NO: 21 - DNA, 20 bp, Artificial Sequence
  Description of Artificial Sequence Primer for GAPDH
cgtcatgggtgtgaaccatg

SEQ ID NO: 22 - DNA, 21 bp, Artificial Sequence
  Description of Artificial Sequence Primer for GAPDH
gctaagcagttggtggtgcag

SEQ ID NO: 23 - DNA, 21 bp, Artificial Sequence
  Description of Artificial Sequence Primer for transferrin receptor
gtcgctggtcagttcgtgatt

SEQ ID NO: 24 - DNA, 23 bp, Artificial Sequence
  Description of Artificial Sequence Primer for transferrin receptor
agcagttggctgttgtacctctc